SEQUENCE LISTING (4 sequences)

SEQ ID NO: 1 - protein, 1408 aa, synthetic construct
  REGION 1..1408: note = artificial screening template for PCT genes
MEGGDIYRASSSRRSSSIWRNSGMEVFSRSSREEDDEEALKWAALEKLPTYDRLRKGILTGSEGEANEVDIKNLGLQERKNLLERLVKVAEEDNEKFLLKLKNRIDRVGIDLPTIEVRFEHLNVEAEAYVGSRALPTFFNFFVNIVEGFLNYLHILPSRKKPLTILHDVSGIIKPGRMTLLLGPPSSGKTTLLLALAGKLDKDLKVSGRVTYNGHGMNEFVPQRTAAYISQHDLHIGEMTVRETLAFSARCQGVGSRYDMLAELSRREKEANIKPDPDIDIYMKAAATEGQEANVVTDYILKILGLEICADTMVGDEMIRGISGGQKKRVTTGEMLVGPAKALFMDEISTGLDSSTTFQIVNSLRQSVHILNGTAVISLLQPAPETYDLFDDIILLSDGQIVYQGPRENVLEFFESMGFKCPERKGVADFLQEVTSRKDQEQYWARRDEPYRFVTVKEFAEAFQSFHVGRKLGDELATPFDKSKSHPAALTTKKYGVSKKELLKACFSREYLLMKRNSFVYIFKLFQLIIMALITMTLFLRTEMHRDTVEDGGIYMGALFFGLVMIMFNGFSELSMTIAKLPVFYKQRDLLFYPSWAYALPTWILKIPITFVEVAVWVFLTYYVIGFDPNVGRFFKQYLLLLLINQMASGLFRFIAALGRNMIVANTFGSFALLILFALGGFVLSRDDVKKWWIWGYWISPLMYGQNAIAVNEFLGHSWRHVLNTETLGVQVLKSRGFFPEAYWYWIGVGALIGYILLFNFLFTLALTYLNPFGKPQAVISEESLAEKARIVEANQNKKRGMVLPFEPHSITFDDIRYSVDMPQEMKAQGVTEDRLVLLKGVSGAFRPGVLTALMGVSGAGKTTLMDVLAGRKTGGYIEGDITISGYPKKQETFARISGYCEQNDIHSPHVTVYESLLYSAWLRLPPEVDSETRKMFIEEVMELVELTPLREALVGLPGVNGLSTEQRKRLTIAVELVANPSIIFMDEPTSGLDARAAAIVMRTVRNTVDTGRTVVCTIHQPSIDIFEAFDELFLMKRGGEEIYVGPLGRHSCHLIKYFEGIEGVSKIKDGYNPATWMLEVTTSAQELALGVDFAEIYKNSELYRRNKALIKELSTPPPGSKDLYFPTQYSQSFLTQCMACLWKQHWSYWRNPPYTAVRFLFTTFIALMFGTMFWDLGSKRSKQQDLFNAMGSMYAAVLFLGVQNASSVQPVVAVERTVFYRERAAGMYSALPYAFAQVLIEIPYIFVQTVVYGVIVYAMIGFEWTAAKFFWYLFFMYFTLLYFTFYGMMTVAVTPNHNIAAIVSSAFYAIWNLFSGFIIPRPRIPVWWRWYYWACPVAWTLYGLVASQFGDITDTLDDGETVEEFLRSYFGFKHDFLGVVAVVVVGFAVLFAFIFAFSIKAFNFQKR

SEQ ID NO: 2 - protein, 1427 aa, synthetic construct
  REGION 1..1427: note = improved artificial screening template for PCT genes
MDGSDIYKAANSIRLGSLRAGSTRSLRSGSTSVWRNSGMDVFSKSSREEDDEEALKWASLEKLPTFDRLKKGLLFGSTGPSNEVDIDNLGVEDRKHLLDRLVKTADEDNEKFLLKLRARLDRVGIDLPTIEVKFEHVTVEADVNTGSRALPSFINFHIDFFEGLLALFHLLPNSKKHITILDDVSGVIKPSRMTLLLGPPSSGKTTLLLALAGTLAKELKSSGKVTYNGHELHEFVPERTSAYISQNDVHIGEMTVRETLAFSARCQGVGSRYDMLAELSRRERDANIKPDPDIDVFMKAAATEGQEASVVTDYTLKLLGLDICADTMVGDQMIRGISGGQKKRVTTGEMIVGPSKVLLMDEISTGLDSSTTYQIVNSLKQFIHILEGTAVISLLQPAPETYDLFDDIILLTDGKIVYQGPRENVLEFFESMGFKCPERKGVADFLQEVTSKKDQQQYWMRRDEPYRFVTAKEFAEAFQSFHVGRKLADDLATPYDKSKSHPAALTTEKYGLNRKELLKACTDREILLMKRNSFVYIFKLFQLTVMALIALTVFFRTEMHRNDLEDGGLYTGALFFGVVMIMFNGMSEISMTIAKLPVFYKQRDFLFYPSWAYALPSWIIKIPVSFLEAAVWTILTYYVIGFDPNVSRFFKQYLILLLVNQMSSGLFRFIGALGRNMIVANTFGSFALLLVFALGGFILSRDDVKGWWIWGYWSSPMMYAMNGIVVNEFLGHGWRTPLNGTTLGKQVITGRGFFAEAYWYWIAVAALIGFILVFNICFALSLAFLDPFGKAQANVSEDDESDAAVELSSDGDEGSQNKKGMILPFEPHSITFDDVKYSVDMPQEMKDQGVGEDRLLLLKGVSGAFRPGVLTALMGVSGAGKTTLMDVLAGRKTGGYIEGDVKISGYPKKQETFARISGYCEQNDIHSPHVTVYESLLYSAWLRLASDVDEHKRKMFVDEVMDLVELNPLKDALVGLPGVNGLSTEQRKRLTIAVELVANPSIIFMDEPTSGLDARAAAIVMRTVRNTVDTGRTVVCTIHQPSIDIFEAFDELFLMKRGGQELYVGPVGRHSCELIEYFEAIDGVSKIKDGYNPATWMLEVSTSAQELALGVDFTEIYRNSDLYKRNKALIAELGVPRPGTKDLFYPTQYSQSFIVQCVACLWKQRLSYWRNPPYTAVRFVFTTFIGIMFGTMFWDLGGKKDTQQDLVNAMGSMYAAVLFLGVQNASAVQPVVDVERTVFYRERAAGMYSALPYAFAQVLVEIPYVFAQTMVYSVIVYAMIGFEWTAAKFFWYLFFQFCCFLYMTFYGMMTVAITPNANIAAIIAASFYGIFNLFSGFIIPRPSIPVWWRWYYWGNPLAWTIYGMVASQFGDYEDVLSNGETVKAYLDRFFGFKHDFLGAVAGVHIGLVLFFAFIFAYCIRSFNFQKR

SEQ ID NO: 3 - DNA, 41 bp, synthetic construct
  source 1..41: mol_type = other DNA
  misc_feature 1..41: note = forward primer for sunflower PCT gene
gacgacgatgacaagttaatatggatggaagtgacatatac

SEQ ID NO: 4 - DNA, 42 bp, synthetic construct
  source 1..42: mol_type = other DNA
  misc_feature 1..42: note = reverse primer for sunflower PCT gene
cctggatcgactagttcaggctatcttttctggaaattgaag

(19) United States
(12) Patent Application Publication
(10) Pub. No.: US 2024/0384288 A1
(43) Pub. Date: Nov. 21, 2024

(54) COUMARIN TRANSPORTERS AND USES THEREOF

(71) Applicants: BASF PLANT SCIENCE COMPANY GMBH, Ludwigshafen (DE); RWTH AACHEN, Aachen (DE)

(72) Inventors: Uwe Conrath, Aachen (DE); Caspar Langenbach, Aachen (DE); Patrick Schwinges, Aachen (DE); David Spencer, Aachen (DE); Holger Schultheiss, Einbeck (DE); Maren Skrobanek, Uebach-Palenberg (DE)

(21) Appl. No.: 18/693,181

(22) PCT Filed: Sep. 15, 2022
(86) PCT No.: PCT/EP2022/075655

§ 371 Date: Mar. 19, 2024

(30) Foreign Application Priority Data
Sep. 20, 2021 (EP) 21197814.3

(51) Int. Cl. C12N 15/82; A01H 1/04

(52) U.S. Cl. CPC C12N 15/8279; A01H 1/045

(57) ABSTRACT

The present invention relates to genes and materials for improving plant health, preferably against phytopathogenic microorganisms, and/or against coumarin-induced adverse effects on plant health. Furthermore, the invention pertains to methods and uses of such genes and mate-rials for creating correspondingly beneficial plant cells, plant parts and whole plants, and also relates to products obtained from such plants or plant parts.

The present invention relates to genes and materials for improving plant health, preferably when plant is attacked by phytopathogenic microorganisms, and/or for improving plant health against coumarin-induced adverse effects on plant health. Furthermore, the invention pertains to methods and uses of such genes and materials for creating correspondingly beneficial plant cells, plant parts and whole plants, and relates to products obtained from such plants or plant parts.

### BACKGROUND OF THE INVENTION

Plant pathogenic organisms, in particular fungi, can cause severe reductions in crop yield and, in worst cases, lead to famines. Monocultures, in particular, are highly susceptible to an epidemic outbreak of diseases. To date, the pathogenic organisms have been controlled mainly by pesticides. Currently, the possibility of directly modifying the genetic features of a plant or of plant pathogens is also open to man. This opens the opportunity to produce naturally occurring fungicides or antifungal compounds by the plants after infection. Alternatively, those antifungal compounds can be synthesized and applied to plants

Yield is affected by various factors, for example the number and size of the plant organs, plant architecture (for example, the number of branches), number of filled seed or grains, plant vigor, growth rate, root development, utilization of water and nutrients and, especially, the severity of abiotic and biotic stress.

In the past, efforts have been made to create plants that are resistant against biotic stress such as that caused by fungal pathogens. The term “resistance” as used herein refers to an absence or reduction of one or more disease symptoms in a plant caused by a plant pathogen. Resistance generally describes the ability of a plant to prevent, or at least curtail the infestation and colonization by a harmful pathogen. Different mechanisms can be discerned in the naturally occurring resistance, with which the plants fend off colonization by phytopathogenic organisms (Schopfer and Brennicke (1999) Pflanzenphysiologie, Springer Verlag, Berlin-Heidelberg, Germany). In nature, however, resistance is often swiftly overcome because of the rapid evolution, huge genetic variability and high progeny number of pathogens which swiftly evolve new virulent races. This holds true for all pathogenic organisms, including fungi (Neu et al. (2003) American Cytopathol. Society, MPMI 16 No. 7:626-633).

Fungi are found worldwide. Approximately 100,000 fungal species are known to date. Amongst them, rust fungi are of great scientific and economic importance. They can have a complex life cycle with up to five different spore stages (spermatium, aecidiospore, uredospore, teleutospore and basidiospore). Specific infection structures are developed for penetration of the plant. Biotrophic phytopathogenic fungi depend on the metabolism of living plant cells for their nutrition. Examples of biotrophic fungi include many rust fungi, powdery mildew fungi and oomycete pathogens such as those in the Phytophthora or Peronospora genera. Necrotrophic phytopathogenic fungi depend for their nutrition on dead plant cells, e.g. species in the genus Fusarium, Rhizoctonia or Mycosphaerella. In this regard, soybean rust occupies an intermediate position. It penetrates the epidermis directly and the penetrated cell soon becomes necrotic. However, after penetration, the fungus switches to an obligate-biotrophic lifestyle. The subgroup of the biotrophic fungal pathogens which follows essentially such an infection strategy are heminecrotrophic.

The soybean rust-causing fungus Phakopsora pachyrhizi directly penetrates the plant epidermis. After growing through the epidermal cell, the fungus reaches the intercellular space of the mesophyll and the fungus starts spreading throughout the leaf. To acquire nutrients, the fungus penetrates mesophyll cells and develops haustoria inside the mesophyll cells by invaginating the plasma membrane of the mesophyll cell. Phakopsora pachyrhizi is a particularly troubling pathogen as it exhibits an immense variability, thereby overcoming novel plant resistance mechanisms and novel fungicide activities within a few years and sometimes even within one growing season. This is especially true for soybean cultivation in Brazil.

Despite its scientific importance, disease resistance is only of economic value if it leads to increased crop yields or better crop quality (in comparison to susceptible varieties) under disease stress conditions.

In the past, attempts were made to produce or accumulate a class of antifungal endogenous secondary metabolites called coumarins in plants, particularly scopoletin, scopolin, esculetin, isoscopoletin, and scoparone. Genes, materials and methods for the synthesis of coumarins in plants are described in WO2016124515 and WO2020120753, which both are incorporated herein as references. While expression of, for example, scopoletin was highly effective at reducing fungal infections, it was also observed that constitutively strong expression of scopoletin biosynthesis genes can lead to reduced overall plant health and yield, particularly in soybean.

It was thus the object of the invention to provide materials and methods to improve plant disease resistance, particularly in crops, and preferably also reducing the negative impact on overall plant health and/or yield which the means of obtaining said improved pathogen resistance may entail. In particular, it was a preferred object of the invention to provide materials and methods which lead to plant material of heritably improved resistance against fungal pathogens with minimised reduction of overall plant health, wherein resistance preferably is directed against a rust fungus and most preferably a fungus in the genus Phakopsora, Fusarium, Sclerotinia, Alternaria, Corynespora, Cercospora, or Septoria.

### SUMMARY OF THE INVENTION

In a first aspect, the invention provides a plant cell comprising a nucleic acid for expression of a heterologous coumarin transporter (PCT) gene.

In a further aspect the invention provides a plant or plant part comprising a plant cell, wherein the plant cell comprises a nucleic acid for expression of a heterologous coumarin transporter (PCT) gene.

In a further aspect the invention provides a plant progeny obtained by breeding a plant of the present invention, wherein the progeny comprises the heterologous PCT gene.

In another aspect the invention provides a non-propagative plant part or material of a plant or plant part of the present invention, preferably a fermentation product, oil, meal, press cake, pomace, chaff, straw or compost.

In a further aspect the invention provides a Product of a plant, plant part or plant cell of the present invention, wherein the product is obtainable or obtained by
i) collecting a material of said plant, plant part or plant cell, preferably a harvestable plant part and most preferably a plant seed, and
ii) disrupting the collected material, preferably to obtain a fermentation product, oil, meal, press cake, pomace, chaff, straw or compost.

The invention also provides a method for providing or increasing coumarin accumulation capability on a plant surface, comprising mutating a wild-type gene such that in the correspondingly encoded PCT protein
the number of differences between the wild-type gene sequence and the amino acid sequence SEQ ID NO. 2 is reduced, and/or
one or more or all of the following mutations, in the numbering according to SEQ ID NO. 2, are introduced into the wild type gene sequence: X8K, X10A, X31T, X32S, X39M, X40D, X67D, X75F, X76G, X78T, X80P, X81S, X84V, X87D, X88N, X91V, X92E, X95K, X96H, X106D, X111K, X118R, X120L, X125I, X127L, X138T, X142D, X156F, X157H, X160I, X164L, X165L, X170L, X171L, X172P, X174S, X178I, X182D, X212A, X218E, X221S, X224K, X233H, X241S, X308A, X310V, X332Q, X356K, X381Q, X382F, X383L, X387E, X416I, X419Q, X424N, X427E, X456E, X460M, X464E, X472K, X473E, X483V, X485R, X489N, X492A, X493T, X497K, X498S, X508E, X512L, X521C, X522T, X523D, X537I, X541F, X544S, X545V, X553V, X562R, X563D, X564L, X567G, X569L, X571M, X574L, X577G, X578V, X579T, X581I, X625S, X626F, X629A, X633T, X647T, X655I, X658L, X669F, X685S, X688L, X689L, X690L, X692F, X701R, X702D, X714S, X720A, X725A, X731G, X732H, X735R, X736T, X737P, X738L, X739N, X740G, X741S, X742T, X746F, X747S, X748I, X752G, X755A, X756E, X772L, X773V, X783A, X786N, X789G, X791A, X794N, X796S, X799D, X800E, X802D, X803A, X804A, X805V, X808T, X809S, X810R, X811N, X812N, X814G, X816Q, X834D, X836K, X847D, X856L, X900D, X947D, X950E, X951H, X952K, X957V, X958D, X969I, X980N, X1073E, X1080A, X1102T, X1108A, X1115E, X1116I, X1128A, X1131A, X1135V, X1144F, X1158V, X1200K, X1203Q, X1207M, X1215A, X1233D, X1258V, X1262I, X1271V, X1287A, X1296Q, X1297L, X1298C, X1303M, X1312A, X1317A, X1318N, X1321A, X1330I, X1339I, X1340P, X1354G, X1372F, X1374D, X1375V, X1378N, X1384E, X1385Y, X1387D, X1390F, X1400V, X1402G, X1404H, X1405V, X1407L, X1408V, X1409L, X1413F, X1417Y.

In a further aspect the invention provides an automated plant selection method, comprising the steps of
i) obtaining, for each seed of a plurality of seeds, a sample comprising genetic material of a tissue body representative for said seed,
ii) determining the presence of a PCT gene according to any the present invention in the genetic material, and optionally the presence of one or more genes of a metabolic pathway for production of one or more coumarins,
iii) selecting those seed where the determination in step ii) gave a positive result.

And the invention provides a use of a heterologous coumarin transporter for any of:
providing or increasing coumarin accumulation on a plant surface,
reduction, attenuation or inhibition of growth of a phytopathogenic microorganism on a plant surface, and
provision or increase of resistance of plants against infection by a phytopathogenic microorganism and/or parasitic plants.

### BRIEF DESCRIPTION OF THE DRAWINGS

FIG. 1 shows the accumulation of scopoletin in sunflower leaves upon biotic or abiotic elicitation. Detached sunflower leaves were either treated with 1 mg/ml P. pachyrhizi uredospores or ultraviolet radiation (λ=254 nm, 30 min). 0.1% Tween and absence of UV exposure served as controls. Scopoletin was measured using HPLC. (methods described in examples 2, 3 and 9)

FIG. 2 shows that sunflower leaf washes contain coumarins that inhibit germination of P. pachyrhizi. Water droplets were placed on UV-treated, detached sunflower leaves and recollected after 24 hours. (A) secretion of the coumarin scopoletin was induced by UV light (+UV). (B) Germination of 1 mg/ml P. pachyrhizi spores in recollected droplets from sunflower (cv. Ames) and soybean (control) leaf surfaces. Coumarins secreted onto the leaf surface of sunflower plants inhibit germination of P. pachyrhizi spores.

FIG. 3 shows that genes with a role in coumarin biosynthesis and transport are induced and coexpressed in sunflower after inoculation. Detached sunflower leaves were treated with 1 mg/ml P. pachyrhizi uredospores or 0.1% Tween. Expression of the HaF6′H1 and HaABC genes relative to the reference gene HaACTIN (phytozome accession number HanXRQChr14g0446641) is shown.

FIG. 4 proves efficient export of scopoletin in Nicotiana tabacum BY-2 suspension cell culture by expression of HaABC. Wildtype, AtF6′H1-expressing and AtF6′H1 plus HaABC-coexpressing BY-2 cells were fed with ferulic acid (which is metabolized by the F6′H1 enzyme to scopoletin). After 0, 2, 4 and 6 hours the intracellular (inserted graph) and extracellular (large graph) scopoletin content was calculated relative to the concentration at the beginning (0 h) of the experiment. Expression of the F6′H1 gene alone leads mainly to accumulation of intracellular scopoletin (inserted graph in the middle), whereas the co-expression of HaABC leads to efficient export of scopoletin (right graph) and to strong reduction of intracellular scopoletin concentration (inserted graph on the right). These findings disclosed HaABC as an efficient transporter for scopoletin.

FIG. 5 discloses the broad substrate range of the HaABC PCT protein and proves that the already published coumarin transporter AtPDR9 (Uniprot: AB37G_ARATH) is unable to transport scopoletin or scoparone. The sunflower transporter gene HaABC (control: AtPDR9) was transiently coexpressed with different coumarin biosynthesis genes in leaves of N. benthamiana plants. Basal scopoletin (A, C) and scoparone (B, D) accumulation in leaf wash water (LWW) was observed upon expression of the biosynthesis genes F6′H1 or F6′H1 plus OMT3, respectively (without transporter). Upon coexpression of the coumarin transporter HaABC DNA construct the content of both coumarins in the leaf wash water (LWW) strongly increased, indicating transport of both coumarins onto the leaf by HaABC (A, B). Note that upon coexpression with the already published AtPDR9 transporter, no increased coumarin secretion was found (C, D).

FIG. 6 shows that heterologous expression of HaABC in N. benthamiana leads to secretion of scopoletin to the leaf surface. The sunflower transporter gene HaABC was expressed in leaves of scopoletin-hyperaccumulating N. benthamiana plants (AtF6′H1-overexpressing background that accumulates scopoletin constitutively).
(A) overexpression of the transporter (driven by the strong constitutive CaMV35S promoter) resulted in five-fold increase of extracellular scopoletin compared to the empty vector Agroinfiltration control (p19). (B), conditional expression of the transporter (by using a DEX-inducible GVG promoter) boosted the overall content of coumarins in leaves after application of DEX (most likely by accumulation on the leaf surface and in the apoplastic space). This indicates that plants are able to accumulate high concentrations of coumarins outside of the cells. (C), germination nation of 1 mg/ml P. pachyrhizi spores in leaf washes (recollected droplets 24 h past treatment) of N. benthamiana expressing HaABC in comparison with the p19 Agroinfiltration control. The findings clearly show that heterologous expression of HaABC in planta leads to an accumulation of coumarins on the leaf surface, where the accumulation of coumarins leads to lower spore germination and, therefore, higher resistance to fungi.

FIG. 7 shows a sequence alignment of SEQ ID NO. 1 and the sequence according to Uniprot entry A0A251UOR5_HELAN. Numbers are given according to the position of Uniprot entry A0A251UOR5_HELAN sequence (label: “PCT”). The number of asterisks above each amino acid of the A0A251UOR5_HELAN sequence indicates the degree of conservation, wherein higher number of stars indicate a stronger conservation. Amino acids given below each amino acid of the A0A251UOR5_HELAN sequence are those of SEQ ID NO. 1 (label: “SEQ. 1”), amino acids further below indicate potential substitutions allowable at the respective position, wherein “-” indicates a gap (deletion relative to the A0A251UOR5_HELAN sequence). The possible substitutions are listed in the order of their respective preference, wherein a more preferred substitution is indicated closer to the respective position in SEQ ID NO. 1.

FIG. 8 shows an alignment of the protein sequences of SEQ ID NO. 1, SEQ ID NO. 2, and the already published genes A0A251UOR5_HELAN, A0A251U1A7_HELAN, A0A5N6LHL1_9ASTR, A0A251U1Q8_HELAN, A0A6S7LXJ0_LACSI, A0A2J6KQ56_LACSA, A0A2J6MG20_LACSA, A0A2U1KVW9_ARTAN, A0A119LIU9_ARTAN, A0A2G2ZJV1_CAPAN, A0A210W665_9ASPA and A0A1S3XDP1_TOBAC. All amino acids are given for SEQ ID NO. 1. For each further sequence, only those amino acids are shown that differ at the respective position from the corresponding amino acid of SEQ ID NO. 1, all amino acids matching the respective amino acid at the respective position of SEQ ID NO. 1 are indicated by “.”. A “-” sign in any sequence indicates an insertion at the respective position in another sequence. Numbering is according to the positions of SEQ ID NO. 1. Soybean sequences are included as negative examples.

### BRIEF DESCRIPTION OF THE SEQUENCES

| SEQ ID NO. | description |
|---|---|
| 1 | artificial screening template for PCT genes |
| 2 | specific artificial screening template for PCT genes |
| 3 | forward primer for sunflower PCT gene |
| 4 | reverse primer for sunflower PCT gene |

### DETAILED DESCRIPTION OF THE INVENTION

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind the skilled person understands that the subject matter of the invention is the sum of the individual technical concepts signified herein or expressed, necessarily in a pars-pro-toto way, by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention.

In so far as recourse herein is made to entries in public databases, for example Uniprot, InterPro and PFAM, the contents of these entries are those as of 2021 May 2. Unless stated to the contrary, where the entry comprises a nucleic acid or amino acid sequence information, such sequence information is incorporated herein.

Nucleic acids and amino acids are abbreviated using their standard one- or three-letter abbreviations. Deletions are indicated by “-”, truncations are indicated by “*”. Alterations of amino acids are specified by the position of the alteration in a respective parent sequence.

As used herein, terms in the singular and the singular forms like “a”, “an” and “the” include plural referents unless the content clearly dictates otherwise. Thus, for example, use of the term “a nucleic acid” optionally includes, as a practical matter, many copies of that nucleic acid molecule; similarly, the term “probe” optionally (and typically) encompasses many similar or identical probe molecules. Also as used herein, the word “comprising” or variations such as “comprises” or “comprising” will be understood to imply the inclusion of a stated element, integer or step, or group of elements, integers or steps, but not the exclusion of any other element, integer or step, or group of elements, integers or steps.

As used herein, the term “and/or” refers to and encompasses any and all possible combinations of one or more of the associated listed items, as well as the lack of combinations when interpreted in the alternative (“or”). The term “comprising” also encompasses the term “consisting of”.

The term “about”, when used in reference to a measurable value, for example an amount of mass, dose, time, temperature, sequence identity and the like, refers to a variation of ±0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50% X whilst in other embodiments it may comprise anywhere from 40% to 60% X (i.e., 50%+10%).

As used herein, the term “gene” refers to a biochemical information which, when materialised in a nucleic acid, can be transcribed into a gene product, i.e. a further nucleic acid, preferably an RNA, and preferably also can be translated into a peptide or polypeptide. The term is thus also used to indicate the section of a nucleic acid resembling said information and to the sequence of such nucleic acid (herein also termed “gene sequence”).

Also as used herein, the term “allele” refers to a variation of a gene characterized by one or more specific differences in the gene sequence compared to the wild type gene sequence, regardless of the presence of other sequence differences. Alleles or nucleotide sequence variants of the invention have at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% nucleotide “sequence identity” to the nucleotide sequence of the wild type gene. Correspondingly, where an “allele” refers to the biochemical information for expressing a peptide or polypeptide, the respective nucleic acid sequence of the allele has at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% amino acid “sequence identity” to the respective wild type peptide or polypeptide.

Protein or nucleic acid variants may be defined by their sequence identity when compared to a parent protein or nucleic acid. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A:
AAGATACTG length: 9 bases
Seq B:
GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “-” symbol in the alignment indicates gaps. The number of gaps introduced by alignment within the sequence B is 1. The number of gaps introduced by alignment at borders of sequence B is 2, and at borders of sequence A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the short sequence).

Accordingly, the alignment length showing sequence A over its complete length would be 9 (meaning sequence A is the sequence of the invention), the alignment length showing sequence B over its complete length would be 8 (meaning sequence B is the sequence of the invention).

After aligning the two sequences, in a second step, an identity value shall be determined from the alignment. Therefore, according to the present description the following calculation of percent-identity applies:

%−identity=(identical residues/length of the alignment region which is showing the respective sequence of this invention over its complete length)*100. Thus, sequence identity in relation to comparison of two amino acid sequences according to the invention is calculated by dividing the number of identical residues by the length of the alignment region which is showing the respective sequence of this invention over its complete length. This value is multiplied with 100 to give “%−identity”. According to the example provided above, %−identity is: for sequence A being the sequence of the invention (6/9)*100=66.7%; for sequence B being the sequence of the invention (6/8)*100=75%.

The term “nucleic acid construct” as used herein refers to a nucleic acid molecule, either single- or double-stranded, which is isolated from a naturally occurring gene or is modified to contain segments of nucleic acids in a manner that would not otherwise exist in nature or is synthetic.

The term “nucleic acid construct” is synonymous with the term “expression cassette” when the nucleic acid construct contains the control sequences required for expression of a polynucleotide.

The term “control sequence” or “genetic control element” is defined herein to include all sequences affecting the expression of a polynucleotide, including but not limited thereto, the expression of a polynucleotide encoding a polypeptide. Each control sequence may be native or foreign to the polynucleotide or native or foreign to each other. Such control sequences include, but are not limited to, promoter sequence, 5′-UTR (also called leader sequence), ribosomal binding site (RBS), 3′-UTR, and transcription start and stop sites.

The term “functional linkage” or “operably linked” with respect to regulatory elements is to be understood as meaning the sequential arrangement of a regulatory element (including but not limited thereto a promoter) with a nucleic acid sequence to be expressed and, if appropriate, further regulatory elements (including but not limited thereto a terminator) in such a way that each of the regulatory elements can fulfil its intended function to allow, modify, facilitate or otherwise influence expression of said nucleic acid sequence. For example, a control sequence is placed at an appropriate position relative to the coding sequence of the polynucleotide sequence such that the control sequence directs the expression of the coding sequence of a polypeptide.

A “promoter” or “promoter sequence” is a nucleotide sequence located upstream of a gene on the same strand as the gene that enables that gene's transcription. A promoter is generally followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

As used herein, the term “isolated DNA molecule” refers to a DNA molecule at least partially separated from other molecules normally associated with it in its native or natural state. The term “isolated” preferably refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods well known to those skilled in the art can be used to isolate and manipulate a polynucleotide, or fragment thereof, as disclosed herein. For example, polymerase chain reaction (PCR) technology can be used to amplify a particular starting polynucleotide molecule and/or to produce variants of the original molecule. Polynucleotide molecules, or fragment thereof, can also be obtained by other techniques, such as by directly synthesizing the fragment by chemical means, as is commonly practiced by using an automated oligonucleotide synthesizer. A polynucleotide can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments of the method include those wherein the polynucleotide is at least one selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used.

As used herein, “recombinant” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if (a) that nucleotide sequence is present in a context other than its natural one, for example by virtue of being (i) cloned into any type of artificial nucleic acid vector or (ii) moved or copied to another location of the original genome, or if (b) the nucleotide sequence is mutagenized such that it differs from the wild type sequence. The term recombinant also can refer to an organism having a recombinant material, e.g., a plant that comprises a recombinant nucleic acid is a recombinant plant.

The term “transgenic” refers to an organism, preferably a plant or part thereof, or a nucleic acid that comprises a heterologous polynucleotide. Preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been so altered by the presence of heterologous nucleic acid including those transgenic organisms or cells initially so altered, as well as those created by crosses or asexual propagation from the initial transgenic organism or cell. A “recombinant” organism preferably is a “transgenic” organism. The term “transgenic” as used herein is not intended to encompass the alteration of the genome (chromosomal or extra-chromosomal) by conventional plant breeding methods (e.g., crosses) or by naturally occurring events such as, e.g., self-fertilization, random cross-fertilization, non-recombinant viral infection, non-recombinant bacterial transformation, non-recombinant transposition, or spontaneous mutation.

As used herein, “mutagenized” refers to an organism or nucleic acid thereof having alteration(s) in the biomolecular sequence of its native genetic material as compared to the sequence of the genetic material of a corresponding wildtype organism or nucleic acid, wherein the alteration(s) in genetic material were induced and/or selected by human action. Examples of human action that can be used to produce a mutagenized organism or DNA include, but are not limited, to treatment with a chemical mutagen such as EMS and subsequent selection with herbicide(s); or by treatment of plant cells with x-rays and subsequent selection with herbicide(s). Any method known in the art can be used to induce mutations. Methods of inducing mutations can induce mutations in random positions in the genetic material or can induce mutations in specific locations in the genetic material (i.e., can be directed mutagenesis techniques), such as by use of a genoplasty technique. In addition to unspecific mutations, according to the invention a nucleic acid can also be mutagenized by using mutagenesis means with a preference or even specificity for a particular site, thereby creating an artificially induced heritable allele according to the present invention. Such means, for example site specific nucleases, including for example zinc finger nucleases (ZFNs), meganucleases, transcription activator-like effector nucleases (TALENS) (Malzahn et al., Cell Biosci, 2017, 7:21) and clustered regularly interspaced short palindromic repeats/CRISPR-associated nuclease (CRISPR/Cas) with an engineered crRNA/tracr RNA (for example as a single-guide RNA, or as modified crRNA and tracrRNA molecules which form a dual molecule guide), and methods of using this nucleases to target known genomic locations, are well known in the art (see reviews by Bortesi and Fischer, 2015, Biotechnology Advances 33:41-52; and by Chen and Gao, 2014, Plant Cell Rep 33:575-583, and references within).

As used herein, a “genetically modified organism” (GMO) is an organism whose genetic characteristics contain alteration(s) that were produced by human effort causing transfection that results in transformation of a target organism with genetic material from another or “source” organism, or with synthetic or modified-native genetic material, or an organism that is a descendant thereof that retains the inserted genetic material. The source organism can be of a different type of organism (e.g., a GMO plant can contain bacterial genetic material) or from the same type of organism (e.g., a GMO plant can contain genetic material from another plant).

As used herein, “wild type” or “corresponding wildtype plant” means the typical form of an organism or its genetic material, as it normally occurs, as distinguished from e.g. mutagenized and/or recombinant forms. Similarly, by “control cell”, “wildtype” “control plant, plant tissue, plant cell or host cell” is intended a plant, plant tissue, plant cell, or host cell, respectively, that lacks the particular polynucleotide of the invention that are disclosed herein. The use of the term “wildtype” is not, therefore, intended to imply that a plant, plant tissue, plant cell, or other host cell lacks recombinant DNA in its genome, and/or does not possess fungal resistance characteristics that are different from those disclosed herein.

As used herein, “descendant” refers to any generation plant. A progeny or descendant plant can be from any filial generation, e.g., F1, F2, F3, F4, F5, F6, F7, etc. In some embodiments, a descendant or progeny plant is a first, second, third, fourth, fifth, sixth, seventh, eighth, ninth, or tenth generation plant.

The term “plant” is used herein in its broadest sense as it pertains to organic material and is intended to encompass eukaryotic organisms that are members of the taxonomic kingdom plantae, examples of which include but are not limited to monocotyledon and dicotyledon plants, vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, underground stems, clumps, crowns, bulbs, corms, tubers, rhizomes, plants/tissues produced in tissue culture, etc.). Unless stated otherwise, the term “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A plant cell is a biological cell of a plant, taken from a plant or derived through culture from a cell taken from a plant.

The invention particularly applies to plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, beans, pea, clover, kudzu, lucerne, lentils, lupins, vetches, groundnut, rice, wheat, barley, Arabidopsis, lentil, banana, canola, cotton, potato, maize, sugar cane, alfalfa, sugar beet, sunflower, rapeseed, sorghum, rice, cabbage, tomato, peppers, sugar cane and tobacco.

According to the invention, a plant is cultivated to yield plant material. Cultivation conditions are chosen in view of the plant and may include, for example, any of growth in a greenhouse, growth on a field, growth in hydroculture and hydroponic growth.

The invention provides a plant cell comprising a nucleic acid for expression of a heterologous coumarin transporter (PCT) gene.

Coumarins are antimicrobial phenolic compounds that can act as phytoanticipins or phytoalexins in plants. Coumarins are produced, for example, upon infection, injury, heat treatment, gamma and ultraviolet irradiation. They have been associated with basal, gene-for-gene, and induced resistance to insects, fungi and other microbes, viruses and postharvest decay (Stringlis, I. A., De Jonge, R. & Pieterse, C. M. J. The Age of Coumarins in Plant-Microbe Interactions. Plant Cell Physiol. 60, 1405-1419 (2019); Chen, J., Shen, Y., Chen, C. & Wan, C. Inhibition of key citrus postharvest fungal strains by plant extracts in vitro and in vivo: A review. Plants 8, 1-19 (2019); Venugopala, K. N., Rashmi, V. & Odhav, B. Review on natural coumarin lead compounds for their pharmacological activity. Biomed Res. Int. 2013, (2013)). While in most plants coumarins are produced in the cytoplasm and are only secreted upon iron defiency (in Arabidopsis roots, Fourcroy, P. et al. Involvement of the ABCG37 transporter in secretion of scopoletin and derivatives by Arabidopsis roots in response to iron deficiency. New Phytol. 201, 155-167 (2014)) or disruption of the tissue (in tobacco hypersensitive response; Costet, L., Fritig, B. & Kauffmann, S. Scopoletin expression in elicitor-treated and tobacco mosaic virus-infected tobacco plants. Physiol. Plant. 115, 228-235 (2002)), leaves of the common sunflower (Helianthus annuus L.) can achieve targeted coumarin export to the epidermal surface, inhibiting fungal pathogens such as Alternaria helianthi and Puccinia spec. (Prats, E., Llamas, M. J., Jorrin, J. & Rubiales, D. Constitutive coumarin accumulation on sunflower leaf surface prevents rust germ tube growth and appressorium differentiation. Crop Sci. 47, 1119-1124 (2007)). Sunflower cultivars with increased concentrations of the coumarins scopoletin and ayapin (6,7-[methylenedioxy]coumarin) have enhanced resistance to diesease (Tal, B. & Robeson, D. J. The Metabolism of Sunflower Phytoalexins Ayapin and Scopoletin. Plant Physiol. 82, 167-172 (1986)). In addition to fungal inhibition, decreased susceptibility of sunflower varieties to phytopathogenic oomycetes and parasitic plants was also linked to enhanced coumarin accumulation (Leon, A., Jorrin-novo, J. V & Novo, J. Agronomic Aspects of the Sunflower 7-Hydroxylated Simple Coumarins 7-Hydroxylated Simple Coumarins. (2016); Gascuel, Q. et al. The sunflower downy mildew pathogen Plasmopara halstedii. Mol. Plant Pathol. 16, 109-122 (2015); Serghini, K., Pérez De Luque, A., Castejón-Muñoz, M., Garcia-Torres, L. & Jorrin, J. V. Sunflower (Helianthus annuus L.) response to broomrape (Orobanche cernua Loefl.) parasitism: Induced synthesis and excretion of 7-hydroxylated simple coumarins. J. Exp. Bot. 52, 2227-2234 (2001)).

Although coumarin transporter genes were identified in the past (Fourcroy, P. et al. Involvement of the ABCG37 transporter in secretion of scopoletin and derivatives by Arabidopsis roots in response to iron deficiency. New Phytol. 201, 155-167 (2014); Lefèvre, F. et al. The Nicotiana tabacum ABC transporter NtPDR3 secretes O-methylated coumarins in response to iron deficiency. J. Exp. Bot. 69, 4419-4431 (2018)) the experimental data only supported the export of catecholic coumarins to the rhizosphere in the case of the Arabidopsis thaliana AtABCG37/AtPDR9 and tobacco NtPDR3. Moreover, the action of the transporter proteins was solely linked to iron starvation and/or hypersensitive response (Robe, K. et al. Coumarin accumulation and trafficking in Arabidopsis thaliana: a complex and dynamic process. New Phytol. 229, 2062-2079 (2021)). None of the above genes was shown to enhance the resistance to fungi or allow coumarin accumulation on the primary site of plant-microbe interaction, the leaf surface.

It has now been found that by providing or increasing the expression of a PCT gene, resistance of plants, in particular crop plants, can be increased against phytopathogenic microorganisms without compromising plant health. As described above, expression of the genes required to install a coumarin synthesis pathway in plants with low or no measurable natural coumarin synthesis results in reduced plant health, in particular to browning of leaves, stunted growth and reduced yield. These adverse effects are particularly notable where expression of a coumarin synthesis gene is driven by a constitutively strong promoter. Surprisingly plant health could be restored in such genetically modified plants without abolishing or reducing coumarin biosynthetic activity.

The invention provides a heterologous PCT gene in a plant cell, that is to say, a plant cell comprising an expression cassette containing a PCT gene which is not found in wild-type plants of the same species, and more preferably is not found in wild-type plants of the same genus. To give some examples, the heterologous PCT gene can be a duplicate of a wild-type PCT gene of the same genus or even species but located in a non-wild-type environment, for example as a further copy of the gene in a non-natural location to increase gene dosage, and/or can be operably connected to a non-natural promoter to increase expression, and/or can be derived from genes of a different species. Heterologous PCT genes can according to the invention be provided on extrachromosomal nucleic acids, e.g. plasmids, but preferably are stably integrated into the plant genome.

The PCT gene according to the invention preferably codes for a PCT protein, wherein the PCT protein comprises, in InterPro-nomenclature and in N- to C-terminal sequence:
i) an IPR029481 ABC-transporter, N-terminal domain,
ii) an IPR003439 ABC transporter-like, ATP-binding domain,
iii) an IPR013525 ABC-2 type transporter domain,
iv) an IPR013581 Plant PDR ABC transporter associated domain,
v) an IPR034003 ATP-binding cassette transporter, PDR-like subfamily G, domain 2 domain,
vi) a further IPR013525 ABC-2 type transporter domain.

Such PCT genes code for PCT proteins of approximately 1200-5400 amino acids, with lengths of 1350-1500 amino acids being preferred.

According to the invention the preferred PCT gene codes for a PCT protein which has 20-90%, preferably 60-85%, more preferably 70% to less than 80% sequence identity to SEQ ID NO. 1. It is to be noted that the amino acid sequences of SEQ ID NO. 1 and SEQ ID NO. 2 are hypothetical protein sequences in the form of a consensus sequence and are not guaranteed nor even expected to be a sequence of a functional coumarin transporter. Thus, each sequence according to SEQ ID NO. 1 and SEQ ID NO. 2 is an artificial amino acid sequence specifically constructed as a template for amino acid sequence screening and annealing purposes and can be used for identification of PCT genes independent from the fact that no PCT activity of the polypeptide of SEQ ID NO. 1 or SEQ ID NO. 2 is shown herein. However, the PCT gene conforming to the above preferred specifications results in increased plant health despite scopoletin synthesis at levels far beyond wild-type concentrations even if the wild-type plants were exposed to pathogenic microorganisms.

Further according to the invention, a preferred PCT gene is obtainable or obtained by selecting, from the genome of a plant capable of secreting coumarins, preferably scopoletin and/or ayapin, a gene having 20-90%, preferably 60-85%, more preferably 70% to less than 80% sequence identity to SEQ ID NO. 1. PCT genes can be found in wild-type plants which produce coumarins, preferably scopoletin and/or ayapin, and secrete such coumarins to their leaves, for example after induction due to phytopathogen stress, e.g. attempted infections by a fungus or oomycete, or after induction using UV irradiation or application of salicylic acid or elicitin. Particularly preferred as wild type PCT gene template plants are such which, when their leaves are washed with water (potentially after induction), produce a strongly UV-fluorescent effluent. An example of such observation is given below. Preferred plants comprising a PCT gene belong to the taxonomic families of Apiaceae, Asteraceae, Convolvulaceae, Euphorbiaceae, Fabaceae, Moraceae, Oleaceae, Orchidaceae, Rutaceae and Thymelaeaceae, and even more preferably to any of the following genera, given in ascending order of preference: Dendrobium, Trema, Vitis, Parasponia, Manihot, Solanum, Nicotiana, Capsicum, Cynara, Artemisia, Lactuca, Micania, Helianthus.

Particularly preferred according to the invention are wild type PCT genes coding for PCT proteins of any of the following Uniprot identifiers, given in increasing order of preference: A0A 103YB99_CYNCS, A0A2H5MYY4_CITUN, A0A3Q7IPK0_SOLLC, A0A2H5N0W9_CITUN, A0A0E0H9C4_ORYNI, A0A0E0H9C3_ORYNI, A0A0E0MZ45_ORYRU, A0A0D3ER34_9ORYZ, A0A0E0MZ44_ORYRU, A0A0D3ER33_9ORYZ, A0A4D8ZQR9_SALSN, A0A3S3P4Y0_9MAGN, A0A200R1R9_9MAGN, A0A3Q7GNC0_SOLLC, A0A068U977_COFCA, A0A1R3GHV5_COCAP, A0A4D9B7F6_SALSN, A0A 151T5S1_CAJCA, A0A498IMZ0_MALDO, A0A0D9YBJ4_9ORYZ, A0A0D9YBJ3_9ORYZ, A0A0E0M010_ORYPU, A0A0E0M009_ORYPU, A0A445CPV2_ARAHY, A0A0D9XCM5_9ORYZ, A0A2G5FBI6_AQUCA, A0A0D9XCM4_9ORYZ, A0A445BQG0_ARAHY, A0A200Q7R1_9MAGN, A0A0D3C4K5_BRAOL, M4DL60_BRARP, A0A 164UQM2_DAUCS, A0A1U7WT16_NICSY, A0A0R0EYV9_SOYBN, A0A2K1J7X2_PHYPA, A0A397ZW27_BRACM, V4MI62_EUTSA, D7LIR7_ARALL, A0A0K9NH37_ZOSMR, R0GUA3_9BRAS, A0A218W919_PUNGR, A0A1E5WM64_9POAL, D8RL77_SELML, M0ZQF4_SOLTU, A0A314Z7L1_PRUYE, A0A1P8AW07_ARATH, D8RL93_SELML, M7ZVI9_TRIUA, M0TWK6_MUSAM, M7ZQM9_TRIUA, A0A1E5VZJ3_9POAL, A0A2K3NST6_TRIPR, AOA3L6SIK5_PANMI, WINN73_AMBTC, WINMF1_AMBTC, A0A0E0C4J0_9ORYZ, I1HYM4_BRADI, A0A1S3BBZ3_CUCME, A0A166G330_DAUCS, A0A2I0WPW6_9ASPA, J3LAT1_ORYBR, A0A0K9PNE7_ZOSMR, A0A453F0X6_AEGTS, AB39G_ORYSJ, A0A0E0CHH1_9ORYZ, I1NYG3_ORYGL, A0A3B6EG33_WHEAT, M0SW47_MUSAM, A0A453F0W9_AEGTS, B8ADW1_ORYSI, A0A2T7F3J3_9POAL, J3L1S2_ORYBR, A0A077SOW7_WHEAT, A0A287L2T7_HORVV, A0A287L2Q0_HORVV, K7UR04_MAIZE, I1HPA0_BRADI, C5XXZ1_SORBI, A0A2R6PIW6_ACTCC, A0A3L6SUJ3_PANMI, A0A2P6PUS6_ROSCH, A0A1D6NLT5_MAIZE, I1NPJ3_ORYGL, AB36G_ORYSI, A0A2I0A7U0_9ASPA, A0A4U6W6V4_SETVI, F6HX55_VITVI, AB36G_ORYSJ, A0A371H4D3_MUCPR, A0A1Q3BMD2_CEPFO, C5XXZ0_SORBI, A0A314ZE00_PRUYE, A0A2T7DJY7_9POAL, K3YPA4_SETIT, A0A2I0W665_9ASPA, A0A4U6UGJ3_SETVI, K3XDS6_SETIT, A0A199W202_ANACO, A0A2R6QZI2_ACTCC, A0A199VL31_ANACO, V4UHT1_CITCL, A0A251N189_PRUPE, A0A0A0L875_CUCSA, A0A2P6PY20_ROSCH, V4ULV4_CITCL, A0A1S3C903_CUCME, A0A061GNG0_THECC, A0A067GW95_CITSI, A0A-A-LNI2_CUCSA, A0A1U8PJ73_GOSHI, V4KC11_EUTSA, A0A0D2UL56_GOSRA, AB40G_ARATH, A0A078HMC6_BRANA, A0A397YWW6_BRACM, A0A0D3CAJ0_BRAOL, D7KDG8_ARALL, A0A078HDZ5_BRANA, M4EBG5_BRARP, A0A1Q3BMC3_CEPFO, A0A251PWV6_PRUPE, A0A2P5CA91_PARAD, A0A0D2N5Y3_GOSRA, A0A059CSN0_EUCGR, A0A2G5FBR3_AQUCA, A0A059BSU9_EUCGR, A0A0K9QMJ0_SPIOL, A0A3S3NY47_9MAGN, A0A067ET34_CITSI, W9SFJ8_9ROSA, A0A1J7IRV6_LUPAN, A0A067KXJ6_JATCU, A0A0K9QP53_SPIOL, B9SMW3_RICCO, W9RAS8_9ROSA, A0A061GHS8_THECC, A0A022QMN3_ERYGU, A0A0L9TA75_PHAAN, A0A067KYQ7_JATCU, A0A1U7ZLW7_NELNU, A0A1U8JF99_GOSHI, A0A2P5EV91_TREOI, B9RGL9_RICCO, A0A2G2VIG6_CAPBA, A0A2K2BSH9_POPTR, A0A_1U7Z913_NELNU, V7BXG5_PHAVU, G7IMF2_MEDTR, A0A022QRS5_ERYGU, A0A1S3U5I5_VIGRR, A0A0S3SFJ6_PHAAN, A0A2K2BSI3_POPTR, A0A2G9GGE9_9LAMI, A0A0L9UPR1_PHAAN, A0A2G9GNR2_9LAMI, A0A445GMB1_GLYSO, A0A0S3SFE0_PHAAN, V7C016_PHAVU, A0A1S3U419_VIGRR, A0A2C9W1E4_MANES, A0A445IF59_GLYSO, G7IMF4_MEDTR, A0A1S2XBA1_CICAR, A0A068TRW1_COFCA, A0A1J7I2U2_LUPAN, A0A1S2XEH4_CICAR, A0A2I4DVL3_JUGRE, A0A371F970_MUCPR, A0A214F7K6_JUGRE, AOA151SU42_CAJCA, I1MCJ9_SOYBN, A0A2P5EV50_TREOI, F6HX68_VITVI, A0A2P5CZE6_PARAD, A0A2C9UZ08_MANES, M1B064_SOLTU, A0A1J6JAR8_NICAT, AOA1S4ANE4_TOBAC, A0A1U7Y8S2_NICSY, A0A1S4AB08_TOBAC, A0A1J6IWB4_NICAT, A0A2G3CRM6_CAPCH, A0A2G2W1J3_CAPBA, A0A2G3CHT0_CAPCH, A0A2G2ZTV6_CAPAN, A0A2U1MPZ3_ARTAN, A0A2G2ZJV1_CAPAN, A0A103XW91_CYNCS, A0A2U1KVW9_ARTAN, A0A2J6MG20_LACSA, A0A6S7LXJ0_LACSI, A0A2J6KQ56_LACSA, A0A5N6LHL1_9ASTR, A0A251U1A7_HELAN, A0A251U0R5_HELAN,
more preferred, again in increasing order of preference, W9SFJ8_9ROSA, A0A1J7IRV6_LUPAN, A0A0K9QP53_SPIOL, W9RAS8_9ROSA, A0A022QMN3_ERYGU, A0A0L9TA75_PHAAN, A0A067KYQ7_JATCU, A0A2G2VIG6_CAPBA, V7BXG5_PHAVU, G7IMF2_MEDTR, A0A022QRS5_ERYGU, A0A1S3U5I5_VIGRR, A0A0S3SFJ6_PHAAN, A0A2G9GGE9_9LAMI, A0A0L9UPR1_PHAAN, A0A2G9GNR2_9LAMI, A0A0S3SFE0_PHAAN, V7C016_PHAVU, A0A1S3U419_VIGRR, G7IMF4_MEDTR, A0A1S2XBA1_CICAR, A0A1J7I2U2_LUPAN, A0A1S2XEH4_CICAR, M1B064_SOLTU, A0A2G3CHT0_CAPCH, A0A2U1MPZ3_ARTAN, A0A2G2ZJV1_CAPAN, AOA103XW91_CYNCS, A0A2U1KVW9_ARTAN, A0A2J6MG20_LACSA, A0A6S7LXJ0_LACSI, A0A2J6KQ56_LACSA, A0A5N6LHL1_9ASTR, A0A251U1A7_HELAN, A0A251U0R5_HELAN
even more preferred, again in increasing order of preference, A0A2G2ZJV1_CAPAN, A0A2U1KVW9_ARTAN, A0A2J6MG20_LACSA, A0A6S7LXJ0_LACSI, A0A2J6KQ56_LACSA, A0A5N6LHL1_9ASTR, A0A251U1A7_HELAN, A0A251UOR5_HELAN,
even more preferred A0A5N6LHL1_9ASTR, A0A251U1A7_HELAN, A0A251UOR5_HELAN, most preferred A0A251U0R5_HELAN (which is also called “HaABC” herein).

Particularly preferred are genes coding for such PCT proteins where the % sequence identity to A0A251U0R5_HELAN (“HaABC”) is preferably at least 59%, more preferably at least 63%, more preferably at least 66%, more preferably at least 73%, more preferably at least 86%, more preferably at least 87%, more preferably at least 88%, more preferably at least 90%, more preferably at least 95% and most preferably is 99-100%. As shown in the examples, the PCT gene of Helianthus annuus is a particularly functional coumarin transporter and can be transferred to other plants, preferably crop plants. Furthermore, after introduction of such heterologous PCT gene adverse effects on plant health by synthesis of coumarins, in particular scopoletin, can be advantageously reduced. The PCT protein preferably differs from the amino acid sequence given by Uniprot identifier A0A251U0R5_HELAN by 0-20 amino acids, more preferably 0-15 amino acids, even more preferably 0-10 amino acids, even more preferably 1-5 amino acids, wherein those differences preferably conform to the constraints according to FIG. 7. If the PCT protein sequence, when aligned to the sequence according to Uniprot identifier A0A251U0R5_HELAN, is longer than said sequence, then each C- or N-terminal extension is preferably no longer than 100 amino acids, more preferably 0-50, even more preferably 0-10 amino acids; likewise, the total length of insertions compared to SEQ ID NO. 2 is preferably 0-100 amino acids, more preferably 0-50 amino acids, more preferably 0-10 amino acids and more preferably 0-5 amino acids.

Preferably the PCT gene codes for a PCT protein which comprises one, more or all of the following mutations, in the numbering according to SEQ ID NO. 2: X8K, X10A, X31T, X32S, X39M, X40D, X67D, X75F, X76G, X78T, X80P, X81S, X84V, X87D, X88N, X91V, X92E, X95K, X96H, X106D, X111K, X118R, X120L, X125I, X127L, X138T, X142D, X156F, X157H, X160I, X164L, X165L, X170L, X171L, X172P, X174S, X178I, X182D, X212A, X218E, X221S, X224K, X233H, X241S, X308A, X310V, X332Q, X356K, X381Q, X382F, X383L, X387E, X416I, X419Q, X424N, X427E, X456E, X460M, X464E, X472K, X473E, X483V, X485R, X489N, X492A, X493T, X497K, X498S, X508E, X512L, X521C, X522T, X523D, X537I, X541F, X544S, X545V, X553V, X562R, X563D, X564L, X567G, X569L, X571M, X574L, X577G, X578V, X579T, X581I, X625S, X626F, X629A, X633T, X647T, X655I, X658L, X669F, X685S, X688L, X689L, X690L, X692F, X701R, X702D, X714S, X720A, X725A, X731G, X732H, X735R, X736T, X737P, X738L, X739N, X740G, X741S, X742T, X746F, X747S, X748I, X752G, X755A, X756E, X772L, X773V, X783A, X786N, X789G, X791A, X794N, X796S, X799D, X800E, X802D, X803A, X804A, X805V, X808T, X809S, X810R, X811N, X812N, X814G, X816Q, X834D, X836K, X847D, X856L, X900D, X947D, X950E, X951H, X952K, X957V, X958D, X969I, X980N, X1073E, X1080A, X1102T, X1108A, X1115E, X1116I, X1128A, X1131A, X1135V, X1144F, X1158V, X1200K, X1203Q, X1207M, X1215A, X1233D, X1258V, X1262I, X1271V, X1287A, X1296Q, X1297L, X1298C, X1303M, X1312A, X1317A, X1318N, X1321A, X1330I, X1339I, X1340P, X1354G, X1372F, X1374D, X1375V, X1378N, X1384E, X1385Y, X1387D, X1390F, X1400V, X1402G, X1404H, X1405V, X1407L, X1408V, X1409L, X1413F, X1417Y. It has been observed that in some cases, wild type PCT genes code for proteins which are insufficient for preventing adverse plant health effects in plants genetically modified to produce coumarins, preferably scopoletin. However, a mutation as listed above sets apart such wild-type genes and increases the coumarin transport capacity of the mutated PCT gene. It is a particular advantage that such coumarin transporters have a broad substrate specificity, as further explained in the examples. Preferably the PCT gene comprises at least 10 of the aforementioned mutations, that is to say, when aligning the PCT gene with the sequence of SEQ ID NO. 2 at least 10 of the aforementioned amino acid positions are conserved. More preferably, the PCT comprises 20-208 of the aforementioned mutations, more preferably 40-200 of the aforementioned mutations, more preferably 80-200 of the aforementioned mutations, more preferably 90-200 of the aforementioned mutations, more preferably 100-200 of the aforementioned mutations. The most preferred PCT gene coding for the protein sequence according to UNIPROT identifier A0A251U0R5_HELAN conforms to these restrictions.

Preferably the plant cell according to the invention is a transgenic plant cell, and preferably the provision of an expression cassette comprising the PCT gene causes the plant cell to be a transgenic one, and/or wherein the PCT gene is operably linked to a heterologous promoter and/or terminator. As described above, the PCT gene is a heterologous PCT gene and can be operably linked to a heterologous promoter and/or terminator. Thus, the invention advantageously provides an easy way of conferring coumarin transporting activity preferably for scopoletin and/or ayapin, to plants lacking such capacity or not having such capacity to an adequate degree to prevent or reduce deleterious plant health effects upon production of coumarins, preferably of scopoletin.

The plant cell according to the invention preferably also comprises a metabolic pathway for production of one or more coumarins. Suitable genes and expression cassettes and methods for introduction of such genes and expression cassettes are in particular described in WO2016124515 and WO2020120753; such genes, expression cassettes and methods for their introduction into plant cells are preferred according to the present invention. Thus, preferred coumarin metabolism genes are F6′H1 (e.g. At3g13610), CCoAOMTI (e.g. At4g34050), ABCG37 (e.g. PDR9; AT3G53480), UGT71 C1 (e.g. At2g29750), 4CL2 and OMT3 and combinations thereof, more preferably F6′H1 and OMT3. Such genes and combinations thereof allow for a particularly suitable production of coumarins, preferably of scopoletin, in plants to prevent, reduce or delay infections by phythopathogenic microorganisms, preferably of rust fungus infections in leguminous plants, most preferably in soybean. Thus, preferably the plant cell of the present invention comprises an expression cassette not only for the PCT gene of the present invention but also a further expression cassette for F6′H1 and preferably also one for OMT3.

Preferably expression of the PCT gene and/or of one or more genes, if present, of the metabolic pathway for production of one or more coumarins is/are regulated such that expression occurs in root, stem and/or leaf cells, and preferably is reduced or repressed in fruit or seed cells. As indicated above, secretion of coumarins had been described in roots, in particular to facilitate iron uptake. However, the present invention provides a way to combat infections by phythopathogenic microorganisms, preferably of fungi or oomycetes. Such infections generally occur via infections of stem or leaf cells. Thus, most preferably expression is regulated to occur not in root cells or only to a lesser degree in root cells compared to stem or leaf cells.

Correspondingly the invention also provides a whole plant or plant part comprising a plant cell of present invention. Such plant or plant part benefits from the advantages imparted by the PCT protein, and in particular is less or not affected by increased scopoletin synthesis and preferably exhibits
coumarin accumulation on a surface of the plant or plant part, and/or
reduced, delayed or inhibited germination or growth of a phytopathogenic microorganism of a surface of the plant or plant part, and/or
increased resistance against infection by a phytopathogenic microorganism and/or increased resistance against parasitic plants.

The pathogenic microorganism according to the invention preferably is selected from any of
phylum Ascomycota, Basidiomycota or Oomycota, more preferably of
order Pleosporales, Heliotiales, Hypocreales or Pucciniales, more preferably of
genus Alternaria, Botrytis, Sclerotinia, Fusarium, or, most preferred, Phakopsora.

Preferred pathogenic microorganisms and the corresponding diseases are also indicated in tables 1 and 2 below.

TABLE 1
Diseases caused by biotrophic and/or heminecrotrophic
phytopathogenic fungi

| Disease | Pathogen |
|---|---|
| Leaf rust | Puccinia recondita |
| Yellow rust | P. striiformis |
| Powdery mildew | Erysiphe graminis/Blumeria graminis |
| Rust (common corn) | Puccinia sorghi |
| Rust (Southern corn) | Puccinia polysora |
| Tobacco leaf spot | Cercospora nicotianae |
| Rust (soybean) | Phakopsora pachyrhizi, P. meibomiae |
| Rust (tropical corn) | Physopella pallescens, P. zeae Angiopsora zeae |

TABLE 2
Diseases caused by necrotrophic and/or hemibiotrophic fungi and oomycetes

| Disease | Pathogen |
|---|---|
| Plume blotch | Septoria (Stagonospora) nodorum |
| Leaf blotch | Septoria tritici |
| Ear fusarioses | Fusarium spp. |
| Late blight | Phytophthora infestans |
| Anthrocnose leaf blight, | Colletotrichum graminicola |
| Anthracnose stalk rot | (teleomorph: Glomerella graminicola Politis); |
| | Glomerella tucumanensis |
| | (anamorph: Glomerella falcatum Went) |
| Curvularia leaf spot | Curvularia clavata, C. eragrostidis, = C. maculans |
| | (teleomorph: Cochliobolus eragrostidis), |
| | Curvularia inaequalis, C. intermedia |
| | (teleomorph: Cochliobolus intermedius), |
| | Curvularia lunata |
| | (teleomorph: Cochliobolus lunatus), |
| | Curvularia pallescens |
| | (teleomorph: Cochliobolus pallescens), |
| | Curvularia senegalensis, C. tuberculata |
| | (teleomorph: Cochliobolus tuberculatus) |
| Didymella leaf spot | Didymella exitalis |
| Diplodia leaf spot | Stenocarpella macrospora = Diplodialeaf macrospora |
| or streak | |
| Brown stripe downy | Sclerophthora rayssiae var. zeae |
| mildew | |
| Crazy top downy mildew | Sclerophthora macrospora = Sclerospora macrospora |
| Green ear downy mildew | Sclerospora graminicola |
| (gramini-cola downy | |
| mildew) | |
| Leaf spots, minor | Alternaria alternata, Ascochyta maydis, A. tritici, A. zeicola, |
| | Bipolaris victoriae = Helminthosporium victoriae, |
| | (teleomorph: Cochliobolus victoriae), |
| | C. sativus |
| | (anamorph: Bipolaris sorokiniana = H. sorokinianum = H. sativum), |
| | Epicoccum nigrum, Exserohilum prolatum = Drechslera prolata |
| | (teleomorph: Setosphaeria prolata), |
| | Graphium penicillioides, Leptosphaeria maydis, Leptothyrium zeae, |
| | Ophiosphaerella herpotricha, |
| | (anamorph: Scolecosporiella sp.), |
| | Paraphaeosphaeria michotii, Phoma sp., Septoria zeae, S. zeicola, S. zeina |
| Northern corn leaf blight | Setosphaeria turcica |
| (white blast, crown stalk | (anamorph: Exserohilum turcicum = Helminthosporium turcicum) |
| rot, stripe) | |
| Northern corn leaf spot | Cochliobolus carbonum |
| Helminthosporium ear rot | (anamorph: Bipolaris zeicola = Helminthosporium carbonum) |
| (race 1) | |
| Phaeosphaeria leaf spot | Phaeosphaeria maydis = Sphaerulina maydis |
| Rostratum leaf spot | Setosphaeria rostrata, |
| (Helminthosporium leaf | (anamorph: xserohilum rostratum = Helminthosporium rostratum) |
| disease, ear and stalk rot) | |
| Java downy mildew | Peronosclerospora maydis = Sclerospora maydis |
| Philippine downy mildew | Peronosclerospora philippinensis = Sclerospora philippinensis |
| Sorghum downy mildew | Peronosclerospora sorghi = Sclerospora sorghi |
| Spontaneum downy | Peronosclerospora spontanea = Sclerospora spontanea |
| mildew | |
| Sugarcane downy mildew | Peronosclerospora sacchari = Sclerospora sacchari |
| Sclerotium ear rot | Sclerotium rolfsii Sacc. |
| (southern blight) | (teleomorph: Athelia rolfsii) |
| Seed rot-seedling blight | Bipolaris sorokiniana, B. zeicola = Helminthosporium carbonum, |
| | Diplodia maydis, Exserohilum pedicillatum, Exserohilum turcicum = |
| | Helminthosporium turcicum, Fusarium avenaceum, F. culmorum, |
| | F. moniliforme, Gibberella zeae |
| | (anamorph: F. graminearum), |
| | Macrophomina phaseolina, Penicillium spp., Phomopsis sp., |
| | Pythium spp., Rhizoctonia solani, R. zeae, Sclerotium rolfsii, |
| | Spicaria sp. |
| Selenophoma leaf spot | Selenophoma sp. |
| Yellow leaf blight | Ascochyta ischaemi, Phyllosticta maydis |
| | (teleomorph: Mycosphaerella zeae-maydis) |
| Zonate leaf spot | Gloeocercospora sorghi |

In particular, the invention provides legumious crop plants, most preferably soybean, comprising the PCT gene as described herein. Such plants, when producing a coumarin, most preferably scopoletin, are preferably protected against infection by a fungal pathogens of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae,
even more preferably of genus Rhizoctonia, Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis,
even more preferably of species Rhizoctonia alpina, Rhizoctonia bicornis, Rhizoctonia butinii, Rhizoctonia callae, Rhizoctonia carotae, Rhizoctonia endophytica, Rhizoctonia floccosa, Rhizoctonia fragariae, Rhizoctonia fraxini, Rhizoctonia fusispora, Rhizoctonia globularis, Rhizoctonia gossypii, Rhizoctonia muneratii, Rhizoctonia papayae, Rhizoctonia quercus, Rhizoctonia repens, Rhizoctonia rubi, Rhizoctonia silvestris, Rhizoctonia solani, Phakopsora ampelopsidis, Phakopsora apoda, Phakopsora argentinensis, Phakopsora cherimoliae, Phakopsora cingens, Phakopsora coca, Phakopsora crotonis, Phakopsora euvitis, Phakopsora gossypii, Phakopsora hornotina, Phakopsora jatrophicola, Phakopsora meibomiae, Phakopsora meliosmae, Phakopsora meliosmae-myrianthae, Phakopsora montana, Phakopsora muscadiniae, Phakopsora myrtacearum, Phakopsora nishidana, Phakopsora orientalis, Phakopsora pachyrhizi, Phakopsora phyllanthi, Phakopsora tecta, Phakopsora uva, Phakopsora vitis, Phakopsora ziziphi-vulgaris, Puccinia abrupta, Puccinia acetosae, Puccinia achnatheri-sibirici, Puccinia acroptili, Puccinia actaeae-agropyri, Puccinia actaeae-elymi, Puccinia antirrhini, Puccinia argentata, Puccinia ar-rhenatheri, Puccinia arrhenathericola, Puccinia artemisiae-keiskeanae, Puccinia arthrocnemi, Puccinia asteris, Puccinia atra, Puccinia aucta, Puccinia ballotiflora, Puccinia bartholomaei, Puccinia bistortae, Puccinia cacabata, Puccinia calcitrapae, Puccinia calthae, Puccinia calthicola, Puccinia calystegiae-soldanellae, Puccinia canaliculata, Puccinia caricis-montanae, Puccinia caricis-stipatae, Puccinia carthami, Puccinia cerinthes-agropyrina, Puccinia cesatii, Puccinia chrysanthemi, Puccinia circumdata, Puccinia clavata, Puccinia coleataeniae, Puccinia coronata, Puccinia coronati-agrostidis, Puccinia coronati-brevispora, Puccinia coronati-calamagrostidis, Puccinia coronati-hordei, Puccinia coronati-japonica, Puccinia coronati-longispora, Puccinia crotonopsidis, Puccinia cynodontis, Puccinia dactylidina, Puccinia dietelii, Puccinia digitata, Puccinia distincta, Puccinia duthiae, Puccinia emaculata, Puccinia erianthi, Puccinia eupatorii-columbiani, Puccinia flavenscentis, Puccinia gastrolobii, Puccinia geitonoplesii, Puccinia gigantea, Puccinia glechomatis, Puccinia helianthi, Puccinia heterogenea, Puccinia heterospora, Puccinia hydrocotyles, Puccinia hysterium, Puccinia impatientis, Puccinia impedita, Puccinia imposita, Puccinia infra-aequatorialis, Puccinia insolita, Puccinia justiciae, Puccinia klugkistiana, Puccinia knersvlaktensis, Puccinia lantanae, Puccinia lateritia, Puccinia latimamma, Puccinia liberta, Puccinia littoralis, Puccinia lobata, Puccinia lophatheri, Puccinia loranthicola, Puccinia menthae, Puccinia mesembryanthemi, Puccinia meyeri-albertii, Puccinia miscanthi, Puccinia miscanthidii, Puccinia mixta, Puccinia montanensis, Puccinia morata, Puccinia morthieri, Puccinia nitida, Puccinia oenanthes-stoloniferae, Puccinia operta, Puccinia otzeniani, Puccinia patriniae, Puccinia pentstemonis, Puccinia persistens, Puccinia phyllostachydis, Puccinia pittieriana, Puccinia platyspora, Puccinia pritzeliana, Puccinia prostii, Puccinia pseudodigitata, Puccinia pseudostriiformis, Puccinia psychotriae, Puccinia punctata, Puccinia punctiformis, Puccinia recondita, Puccinia rhei-undulati, Puccinia rupestris, Puccinia senecionis-acutiformis, Puccinia septentrionalis, Puccinia setariae, Puccinia silvatica, Puccinia stipina, Puccinia stobaeae, Puccinia striiformis, Puccinia striiformoides, Puccinia stylidii, Puccinia substriata, Puccinia suzutake, Puccinia taeniatheri, Puccinia tageticola, Puccinia tanaceti, Puccinia tatarinovii, Puccinia tetragoniae, Puccinia thaliae, Puccinia thlaspeos, Puccinia tillandsiae, Puccinia tiritea, Puccinia tokyensis, Puccinia trebouxi, Puccinia triticina, Puccinia tubulosa, Puccinia tulipae, Puccinia tumidipes, Puccinia turgida, Puccinia urticae-acutae, Puccinia urticae-acutiformis, Puccinia urticae-caricis, Puccinia urticae-hirtae, Puccinia urticae-inflatae, Puccinia urticata, Puccinia vaginatae, Puccinia virgata, Puccinia xanthii, Puccinia xanthosiae, Puccinia zoysiae, more preferably of species Phakopsora pachyrhizi, Puccinia graminis, Puccinia striiformis, Puccinia hordei or Puccinia recondita, more preferably of genus Phakopsora and most preferably Phakopsora pachyrhizi. As indicated above, fungi of these taxa are responsible for grave losses of crop yield. This applies in particular to rust fungi of genus Phakopsora. It is thus an advantage of the present invention that the method allows to reduce fungicide treatments against Phrakopsora pachyrhizi.

The invention also provides plant progeny obtained by breeding a plant according to the invention, wherein the progeny comprises the heterologous PCT gene. Thus, the invention advantageously allows to introduce the trait of coumarin transport capacity and/or reduction or prevention of deleterious plant health effects by coumarin biosynthesis, most preferably of scopoletin biosynthesis, into plant varieties and species naturally devoid of such trait. It is a further advantage that incorporation of the trait can be easily monitored by detecting the presence of the heterologous PCT gene in offspring plant material.

Furthermore, the invention provides a non-propagative plant part or material of a plant or plant part according to the invention, preferably a fermentation product, oil, meal, press cake, pomace, chaff, straw or compost. Such material advantageously can enrich coumarins, e.g. in straw, and is thus particularly suitable to suppress spreading of phythopathogenic microorganisms by depositing the material on and/or in a plant cultivation substrate. Furthermore, the activity of the PCT gene according to the present invention is useful to reduce coumarin-induced deleterious effects on plant health and thus is useful to increase the yield of plant material, which in turn advantageously increases the availability of the aforementioned plant parts, materials or products made using such plant parts or materials.

Correspondingly the invention also provides a product of a plant, plant part or plant cell according to the invention, wherein the product is obtainable or obtained by
i) collecting a material of said plant, plant part or plant cell, preferably a harvestable plant part and most preferably a plant seed, and
ii) disrupting the collected material, preferably to obtain a fermentation product, oil, meal, press cake, pomace, chaff, straw or compost.

Furthermore, the invention provides a method for providing or increasing coumarin accumulation capability on a plant surface, comprising mutating a wild type gene such that in the correspondingly encoded PCT protein
the number of differences between the wild type gene sequence and the amino acid sequence SEQ ID NO. 2 is reduced, and/or
one or more or all of the following mutations, in the numbering according to SEQ ID NO. 2, are introduced into the wild type gene sequence: X8K, X10A, X31T, X32S, X39M, X40D, X67D, X75F, X76G, X78T, X80P, X81S, X84V, X87D, X88N, X91V, X92E, X95K, X96H, X106D, X111K, X118R, X120L, X125I, X127L, X138T, X142D, X156F, X157H, X160I, X164L, X165L, X170L, X171L, X172P, X174S, X178I, X182D, X212A, X218E, X221S, X224K, X233H, X241S, X308A, X310V, X332Q, X356K, X381Q, X382F, X383L, X387E, X416I, X419Q, X424N, X427E, X456E, X460M, X464E, X472K, X473E, X483V, X485R, X489N, X492A, X493T, X497K, X498S, X508E, X512L, X521C, X522T, X523D, X537I, X541F, X544S, X545V, X553V, X562R, X563D, X564L, X567G, X569L, X571M, X574L, X577G, X578V, X579T, X5811, X625S, X626F, X629A, X633T, X647T, X655I, X658L, X669F, X685S, X688L, X689L, X690L, X692F, X701R, X702D, X714S, X720A, X725A, X731G, X732H, X735R, X736T, X737P, X738L, X739N, X740G, X741S, X742T, X746F, X747S, X748I, X752G, X755A, X756E, X772L, X773V, X783A, X786N, X789G, X791A, X794N, X796S, X799D, X800E, X802D, X803A, X804A, X805V, X808T, X809S, X810R, X811N, X812N, X814G, X816Q, X834D, X836K, X847D, X856L, X900D, X947D, X950E, X951H, X952K, X957V, X958D, X969I, X980N, X1073E, X1080A, X1102T, X1108A, X1115E, X1116I, X1128A, X1131A, X1135V, X1144F, X1158V, X1200K, X1203Q, X1207M, X1215A, X1233D, X1258V, X1262I, X1271V, X1287A, X1296Q, X1297L, X1298C, X1303M, X1312A, X1317A, X1318N, X1321A, X1330I, X1339I, X1340P X1354G, X1372F, X1374D, X1375V, X1378N, X1384E, X1385Y, X1387D, X1390F, X1400V, X1402G, X1404H, X1405V, X1407L, X1408V, X1409L, X1413F, X1417Y.

The advantages of such method have already been described above and are furthermore apparent in view of the examples.

Several of the positions of A0A251U0R5_HELAN are pairwise correlated. If the PCT gene codes for a PCT protein wherein an amino acid is exchanged at a first one of the correlated positions relative to A0A251U0R5_HELAN, then a matching amino acid at the respective second one of the correlated positions is chosen. Preferably, the amino acids at the correlated positions are identical to those of A0A251U0R5_HELAN. The correlated positions of A0A251U0R5_HELAN are, in decreasing order of correlation and thus in decreasing order of preferred conservation: 835+861, 934+971, 870+1052, 193+408, 258+326, 837+861, 839+857, 1191+1280, 659+765, 552+640, 860+1061, 837+857, 965+994, 574+643, 1296+1364, 670+778, 515+612, 365+373, 183+416, 975+991, 833+864, 1158+1260, 1000+1033, 937+994 and 656+765.

The invention also provides an automated plant selection method, comprising the steps of
i) obtaining, for each seed of a plurality of seeds, a sample comprising genetic material of a tissue body representative for said seed,
ii) determining the presence of a PCT gene according to the present invention in the genetic material, and optionally the presence of one or more genes of a metabolic pathway for production of one or more coumarins,
iii) selecting those seed where the determination in step ii) gave a positive result.

It is a particular advantage that the invention provides, as described above, a heritable trait whose presence can be easily ascertained in offspring plants even before germination of the plants and exposition to phythopathogenic microorganisms, thereby allowing for a particularly fast breeding process. Such processes can advantageously further be sped up by automatically analysing tissue material representative of an individual seed of a plurality of seeds, e.g. DNA of testa or of seed cells. Thus, seeds that do not contain a functional PCT gene according to the invention can automatically be discarded, thereby reducing the content of non-trait harbouring material in offspring generated by breeding.

In view of the aforementioned effects and advantages, the invention also teaches the use of a heterologous coumarin transporter for any of:
providing or increasing coumarin accumulation on a plant surface,
reduction, attenuation or inhibition of growth of a phytopathogenic microorganism on a plant surface, and
provision or increase of resistance of plants against infection by a phytopathogenic microorganism and/or parasitic plants.

Selected aspects of the invention are hereinafter described in more detail by non-limiting examples.

### EXAMPLES

### Example 1: Material

Phakopsora pachyrhizi was continuously maintained on soybean plants grown in 16-hour light at 24° C. and 8 h dark at 21° C. Three-week-old soybean plants were inoculated with freshly harvested P. pachyrhizi spores (1 mg/mL in water with 0.01% Tween-20) and stored for 24 h in the dark in a humid surrounding. Afterwards, the plants were kept in a growth chamber until approximately 10 dpi uredosori were formed on the abaxial leaf side. This procedure was repeated in a weekly manner. Sunflower and N. benthamiana plants were grown in the same conditions and used for experiments at 5-6 weeks (4-5 weeks for N. benthamiana).

### Example 2: Elicitation of Coumarin Accumulation in Sunflower

Second or third true leaves of 5-week-old sunflower cv. Ames 18925 (PI 650755) were detached and placed in square petri dishes supplemented with moistened paper. Leaves were either sprayed with freshly harvested P. pachyrhizi spores (see above) or treated with shortwave ultraviolet (UV) irradiation (λ=254 nm, q=0.44 μmol m−2 s−1, 30 min, leaves were placed approx. 20 cm from the source).

### Example 3: Coumarin Extraction From Leaf Surfaces

Droplets of double-distilled water (30 μl) were evenly distributed on the surface of detached leaves placed in a petri dish (approx. 40 droplets on sunflower, 20 droplets on N. benthamiana). Samples were kept at RT for 24 h at high humidity. Subsequently, droplets were carefully collected without touching the leaf surface, filtered and used for HPLC analysis or germination assays.

### Example 4: Germination Assays in Plant Leaf Washes

Uredospores of P. pachyrhizi (isolate BR05) were harvested from heavily infected soybean leaves and suspended in water by shaking for 25 s at 5′000 rpm on a Precellys 24 homogenizer in the absence of homogenizing beads (Bertin instruments). For in vitro assays, spores were added to the adequate test solution at a density of 1 mg spores/ml. Using a diffuser (Carl Roth), uredospores were subsequently sprayed on glass slides that were coated with polyethylene foil. The slides were kept for 18 h at high humidity. Germination of spores was evaluated by counting developing germ tubes in the leaf wash water (Example 3) supplemented with 0.01% Tween-20.

### Example 5: Cloning

DNA sequences of all coumarin transporters used were generated by DNA synthesis (Geneart, Regensburg, Germany).

For stable expression in BY-2 cells cDNA sequences of genes of interest (“GOIs”) were cloned in the binary pB2GW7 (Ghent University, Belgium) vector using Gibson assembly® and Ascl and Pacl restriction sites (for A0A251U0R5_HELAN: FW oligo: gacgacgatgacaagttaatATGGATGGAAGTGACATATAC, RV oligo: cctggatcgactagttcaggCTATCTTTTCTGGAAATTGAAG).

For combinatorial expression of GOIs AtF6′H1 and A0A251U0R5_HELAN genes were expressed under control of the same promoter. To ensure translation of the A0A251U0R5_HELAN gene at the second position a ribosomal binding site was integrated at its 5′ end.

For transient in planta overexpression assays cDNA sequences of GOIs were cloned in the plant overexpression vector pB7WG2D13 using the Gateway® cloning technology. Similar cloning steps are performed to generate transient in-planta overexpression constructs for any of the other coumarin transporter proteins A0A251U1Q8_HELAN, A0A5N6LHL1_9ASTR, A0A 1I9L1U9_ARTAN, A0A2J6KQ56_LACSA and A0A2G2ZJV1_CAPAN with A0A1S3XDP1_TOBAC as control.

### Example 6: Precursor Feeding in BY-2 Cell Suspension Cultures

Cell suspension cultures of Nicotiana tabacum BY-2 cells obtained according to Example 5 were grown according to the protocol from Taguchi et al. (Taguchi, G. et al. Scopoletin uptake from culture medium and accumulation in the vacuoles after conversion to scopolin in 2,4-D-treated tobacco cells. Plant Sci. 151, 153-161 (2000)) and subcultured for 7 d. To start the experiment, a 24-well plate was filled with 1 ml of the respective culture and supplemented with 400 μM ferulic acid dissolved in 100% DMSO (control feeding with 10 μl DMSO, i.e. 1% DMSO) and returned to shaking at 190 rpm. After 0, 1, 2, 3 and 6 h, samples were harvested. Cells were centrifuged for 10 min at 8,000×g and the supernatant was analyzed directly via HPLC (“medium scopoletin”). Cells were dried, weighed and ground before extracting coumarins according to the procedure described for plant tissue (see below).

### Example 7: Transient Transformation of N. benthamiana

Transient transformation of N. benthamiana leaves was done according to a slightly modified protocol from Popescu et al. (Popescu, S. C. et al. Differential binding of calmodulin-related proteins to their targets revealed through high-density Arabidopsis protein microarrays. 104, 4730-4735 (2007)). A single Agrobacterium strain (AGL01) carrying a DNA construct of interest was cultured in YEB medium with appropriate antibiotics for 14-16 h at 28° C. Cells were harvested by centrifugation (5000 rpm 10 min), resuspended to an OD of 0.4-0.8 in buffer containing 10 mM MgCl2, 10 mM MES pH 5.6 and 150 mM acetosyringone and incubated for 2-5 h at room temperature. Agrobacteria transformed with the DNA construct of interest were then mixed with an equal volume of Agrobacteria containing the p19 silencing suppressor gene from tomato bushy stunt virus (TBSV) and 1:1 mixtures were syringe-infiltrated into leaves of 5-week-old N. benthamiana plants. Three days after Agrobacterium infiltration, leaves were either used for leaf wash assays or frozen in liquid nitrogen and stored at −80° C. until analysis.

### Example 8: Coumarin Extraction From Plant Tissue

Approximately 500 mg lyophilized and ground leaves were mixed with 1 ml of methanol in a 2 ml reaction tube and vortexed vigorously. Tubes were sealed and incubated overnight at RT with gentle shaking. Subsequently, the samples were centrifuged for 10 min at 21,000 rcf. Seven-hundred microliters of supernatant were transferred to a fresh 2-ml reaction tube and evaporated in a vacuum concentrator. The dry pellet was resuspended in 200 μl methanol and stored at −20° C.

### Example 9: High Performance Liquid Chromatography (HPLC)

Coumarin methanol extracts were centrifuged for 10 min at 21,000 rcf and 70 μl of the supernatant were separated via HPLC on a reverse phase C18 column (KROMAPLUS 100-5-C18 5.0, Prontosil). ddH2O and acetonitrile, both containing 1.5% (v/v) acetic acid, were used as solvents. The pump gradient was as follows: the ratio of acetonitrile started at 10% and increased up to 25% within 10 min in a linear manner. It remained at 25% until minute 20 where it gradually increased up to 90% within 2 min. From minute 22 to minute 25 the proportion of acetonitrile stayed at 90% before it dropped down to 10% during the last 2 min. Chromatography was carried out at a flow rate of 1 ml/min with a run duration of 27 min. Coumarin fluorescence was detected at λ=335 nm excitation and λ=460 nm emission (FP 920 Intelligent Fluorescence Detector, Jasco) or at λ=342 nm (UV absorbance). Coumarin peaks were identified by comparison to retention times of authentic commercial standards. For quantification, a standard curve of commercial standards with known concentrations was measured.

### Example 10: Observations

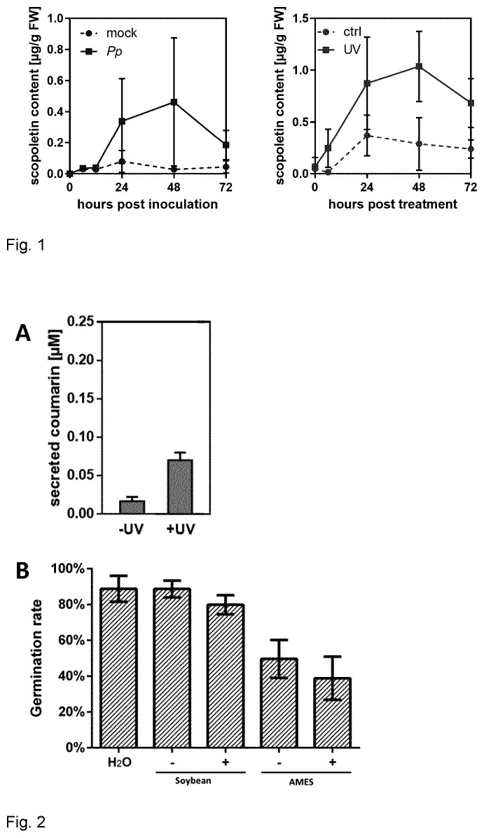

Coumarin secretion of sunflower can be triggered by inoculation with P. pachyrhizi, the causative agent of Asian Soybean Rust disease (SBR) and/or treatment with ultraviolet radiation. Scopoletin content in wild-type sunflower leaves increases after biotic and abiotic elicitation. As described in Example 2, detached sunflower leaves were either treated with 1 mg/ml P. pachyrhizi uredospores (see FIG. 1 left graph) or ultraviolet radiation (λ=254 nm, 30 min; see FIG. 1 right graph). 0.1% Tween and no UV exposure served as controls. Scopoletin content increased after both elicitation events within 24 h by at least 2 fold over the scopoletin content of the respective control and further increased until 48 h after elicitation before slightly dropping.

Water droplets collected from stressed sunflower leaves contain the coumarin scopoletin (see. FIG. 2A). Water droplets were placed on UV-treated, detached sunflower leaves and recollected after 24 hours (leaf wash water) as described in Example 3. Enrichment of the water droplets by scopoletin could be visually detected by blue fluorescence under ultraviolet light.

Germination of P. pachyrhizi in leaf wash water is reduced, and the inhibitory effect increases upon pre-treatment, i.e. induction of the leaves with UV light (see FIG. 2B). As described in example 4, 1 mg/ml P. pachyrhizi spores were germinated in recollected droplets from sunflower and soybean (control) leaf surfaces with and without prior UV elicitation. In UV treated soybean leaves P. pachyrhizi germination was reduced to 80% of the germination probability of pure water or water collected from UV untreated leaves. P. pachyrhizi germination probability in water collected from UV untreated and treated sunflower leaves was reduced, relative to germination probability using pure water, to less than 60% (untreated leaves) and 40% (treated sunflower leaves).

The expression profiles of the scopoletin biosynthesis gene HaF6′H1 and the PCT gene (coding for the protein according to Uniprot identifier A0A251U0R5_HELAN) correlated with coumarin extrusion to the washing fluid (cf. FIG. 3). As described in examples 2 and 3, detached sunflower leaves were treated with 1 mg/ml P. pachyrhizi uredospores or 0.1% Tween (mock).

Expression of (A) F6′H1 (legend: “HaF6′H1”) and the (B) PCT gene (legend: “HaABC”) relative to the reference gene HaACTIN (JGI phytozome accession number HanXRQChr14g0446641) is shown in FIG. 3. Both expression of the F6H1 gene and the PCT gene were induced at 12 h after induction, with induction of the PCT gene being stronger than induction of the F6′H1 gene.

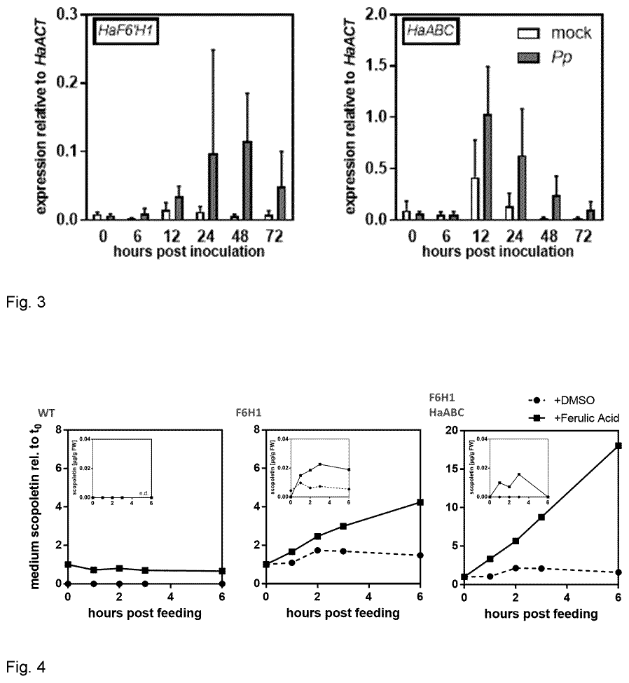

Expression of the PCT gene (coding for the protein according to Uniprot identifier A0A251U0R5_HELAN) in BY-2 cells resulted in marked increase of medium scopoletin content. As described in Example 6 medium scopoletin concentration was measured for media of wildtype (see. FIG. 4A), F6H1-expressing (see FIG. 4B, which shows expression of the F6′H1 gene of A. thaliana, legend: “AtF6H1”) and AtF6′H1/PCT-expressing (see FIG. 4C, legend for the PCT gene coding for A0A251U0R5_HELAN: “HaABC”) BY-2 cells were fed with 400 μM ferulic acid or DMSO (control). After 0, 2, 4 and 6 hours the intra- and extracellular scopoletin content was calculated relative to the concentration at the start (0 h) of the experiment. Within 2 h after the start of feeding, medium concentrations of scopoletin did not change relative to the concentration at the start of feeding for the wild type cells. For the F6H1-expressing cells, media concentrations of scopoletin increased over control at 2 h after the start of feeding and continued to increase linearly until 6 h after start of feeding (end of experiment). This effect was stronger by a factor of 2 in the F6H1- and PCT-gene expressing cells.

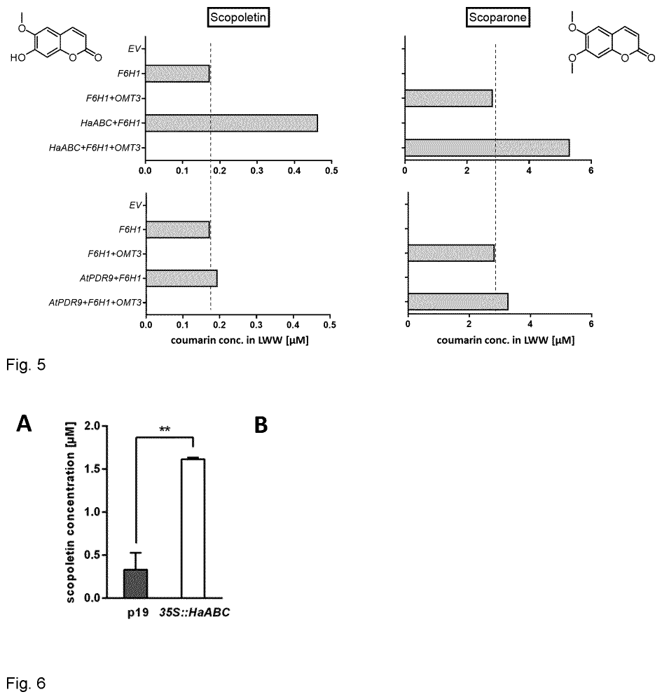

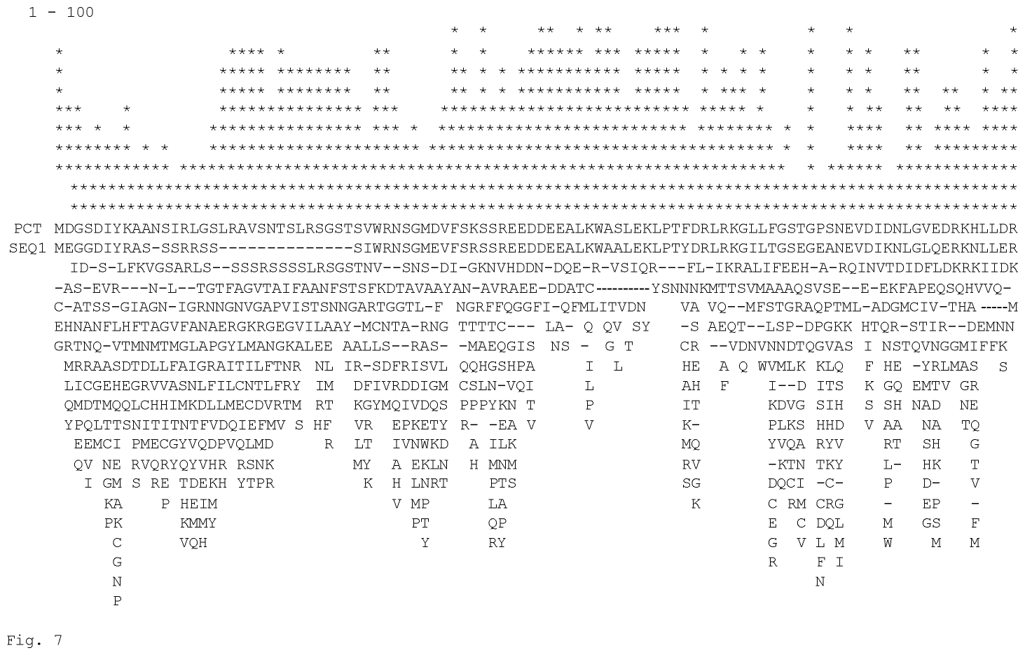

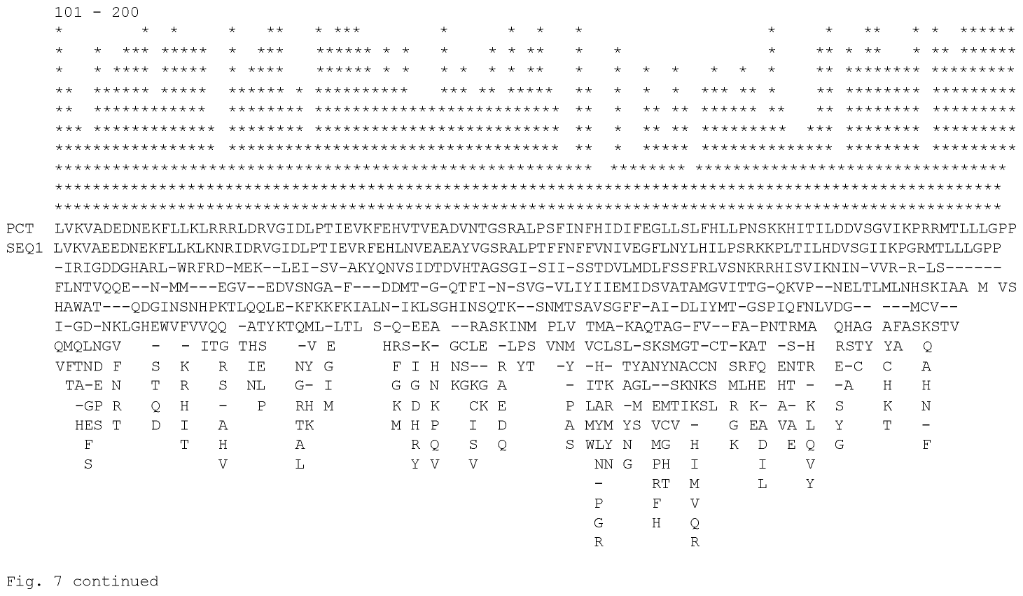

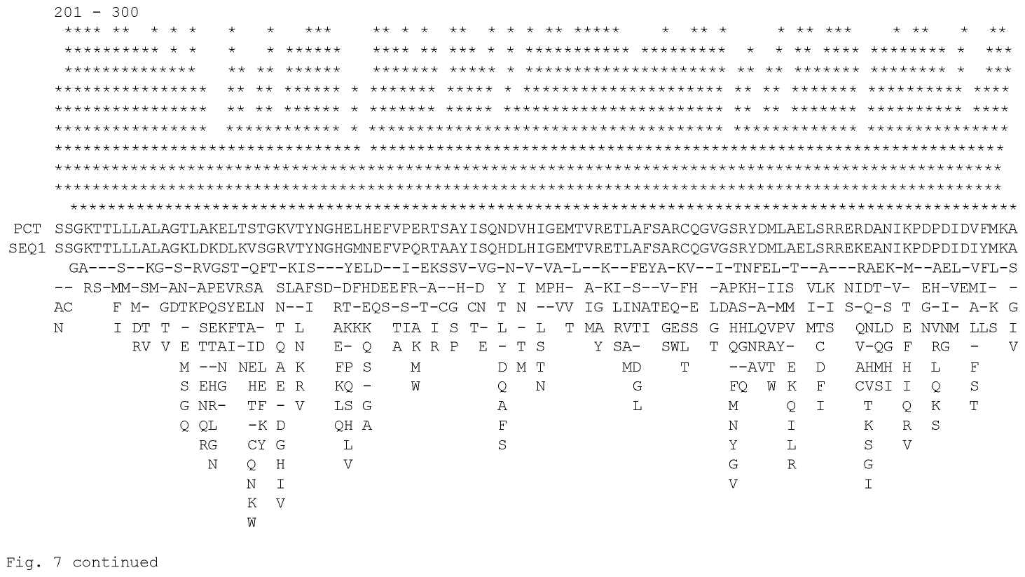

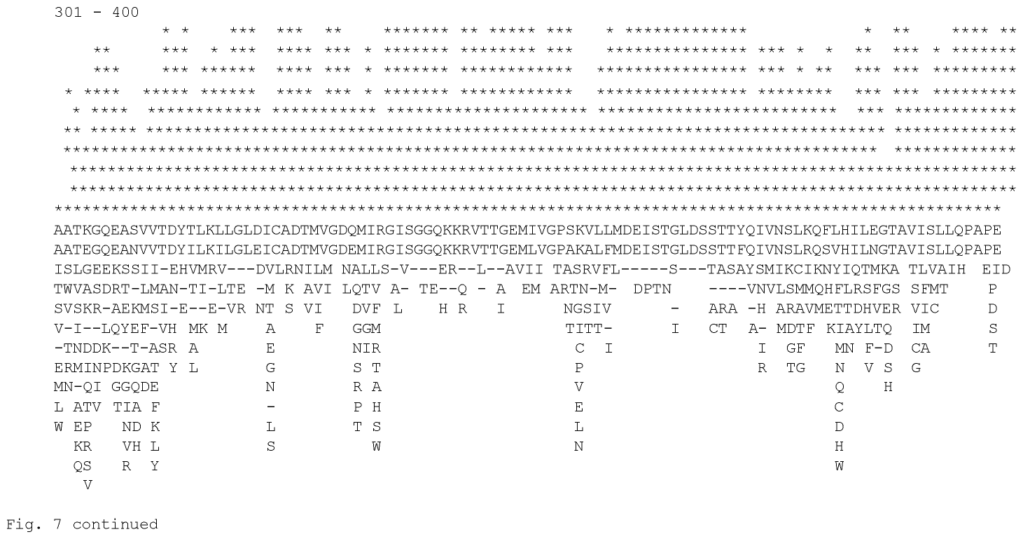

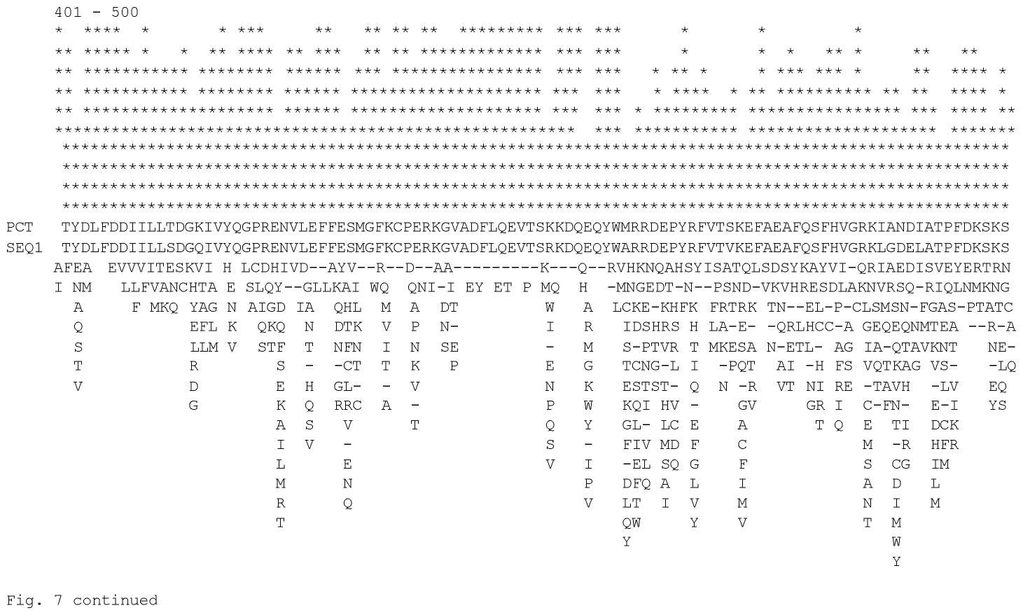

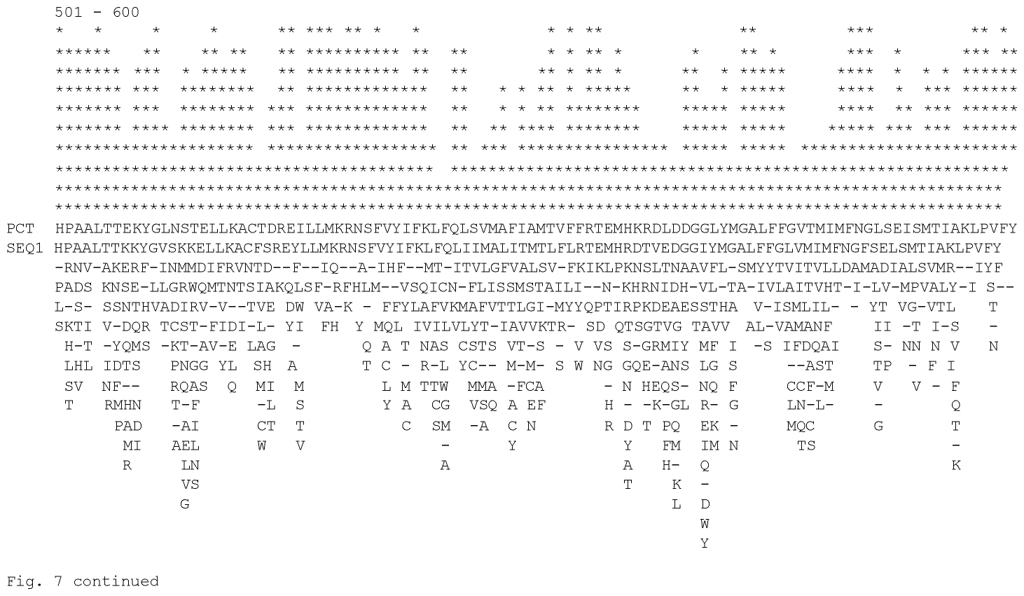

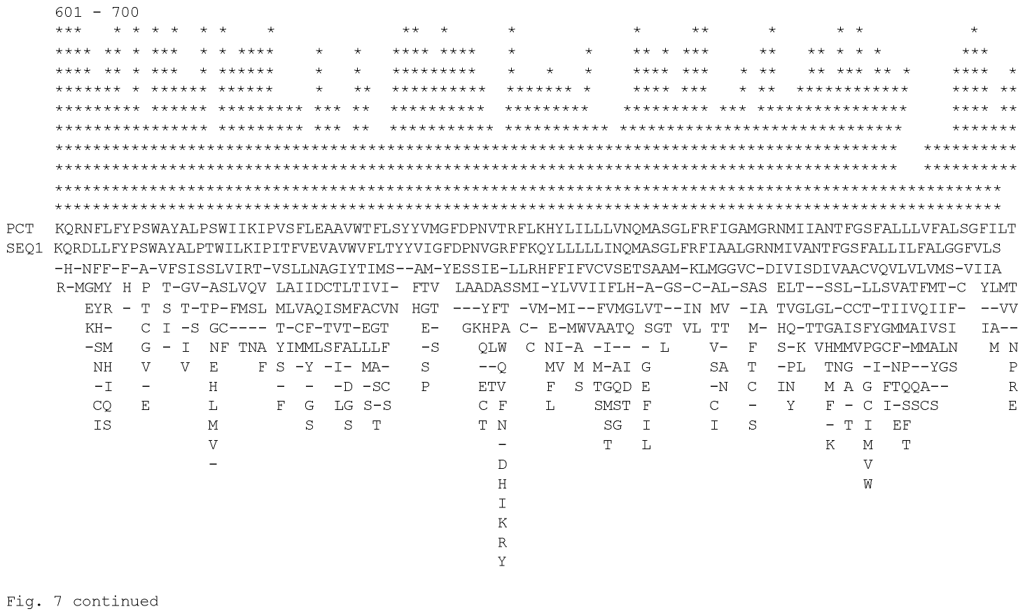

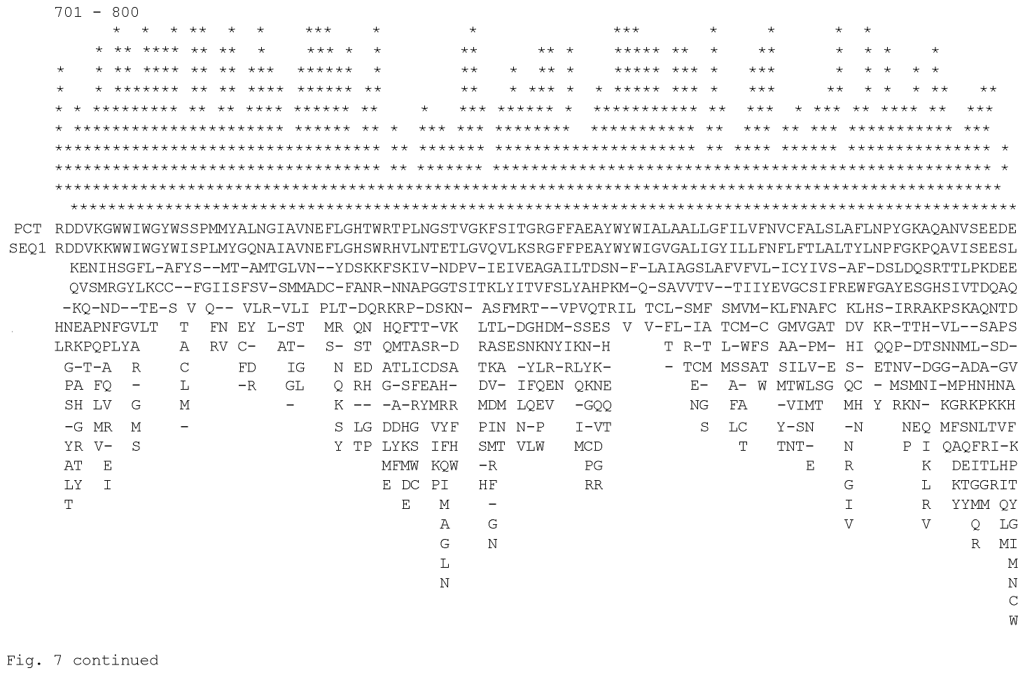

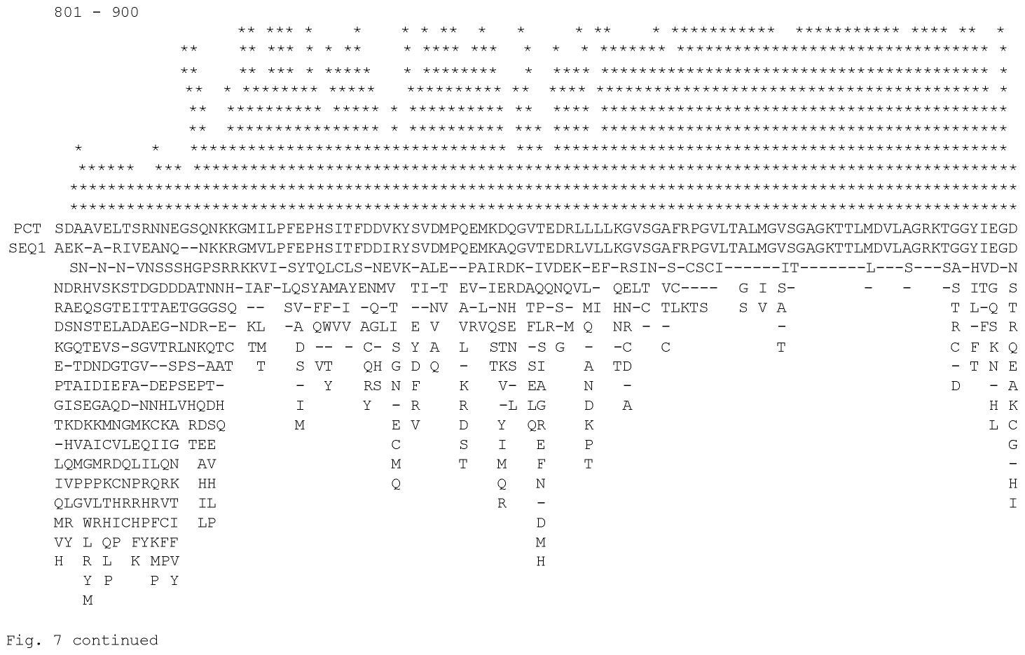

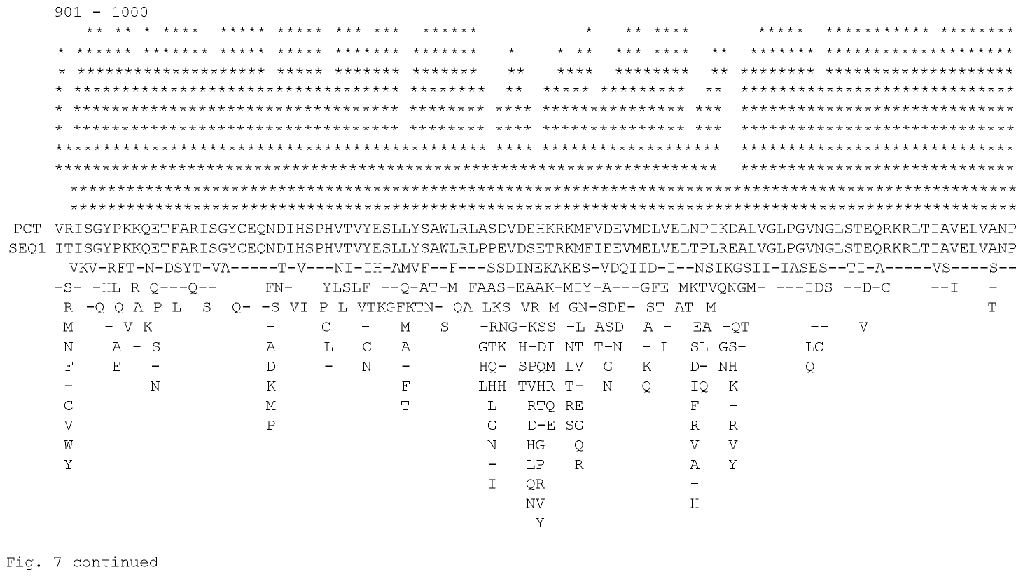

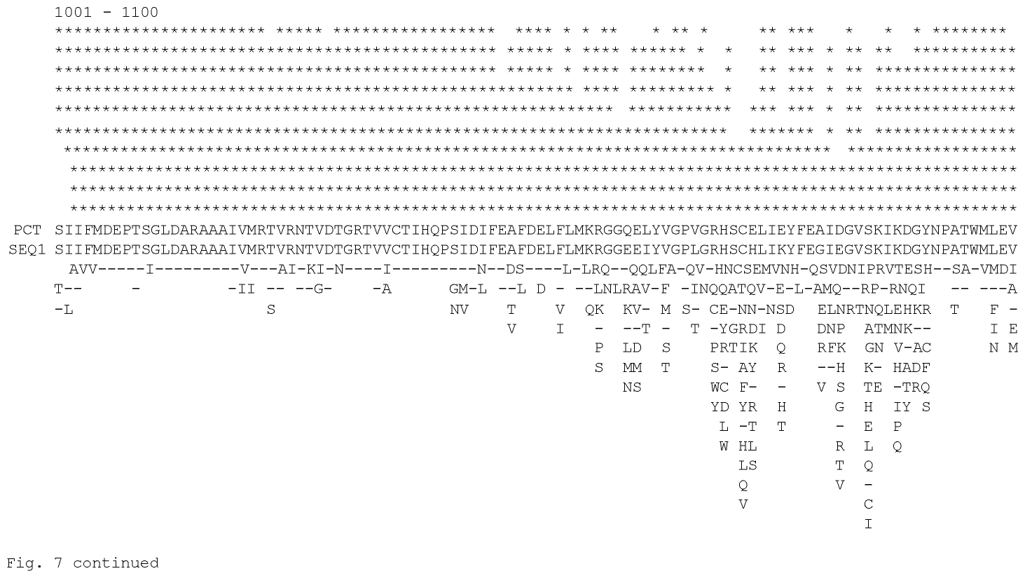

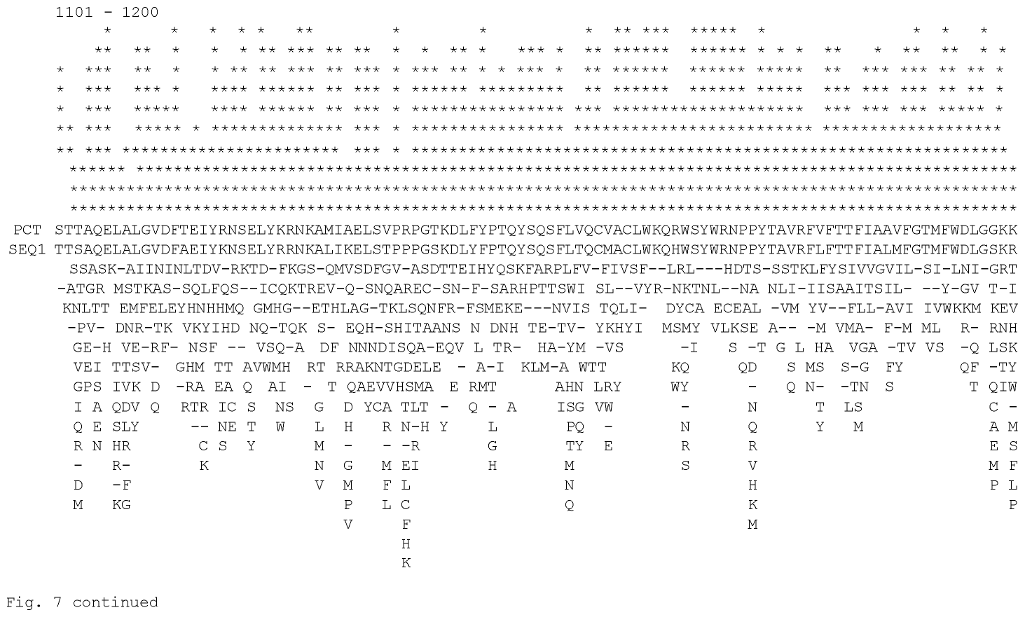

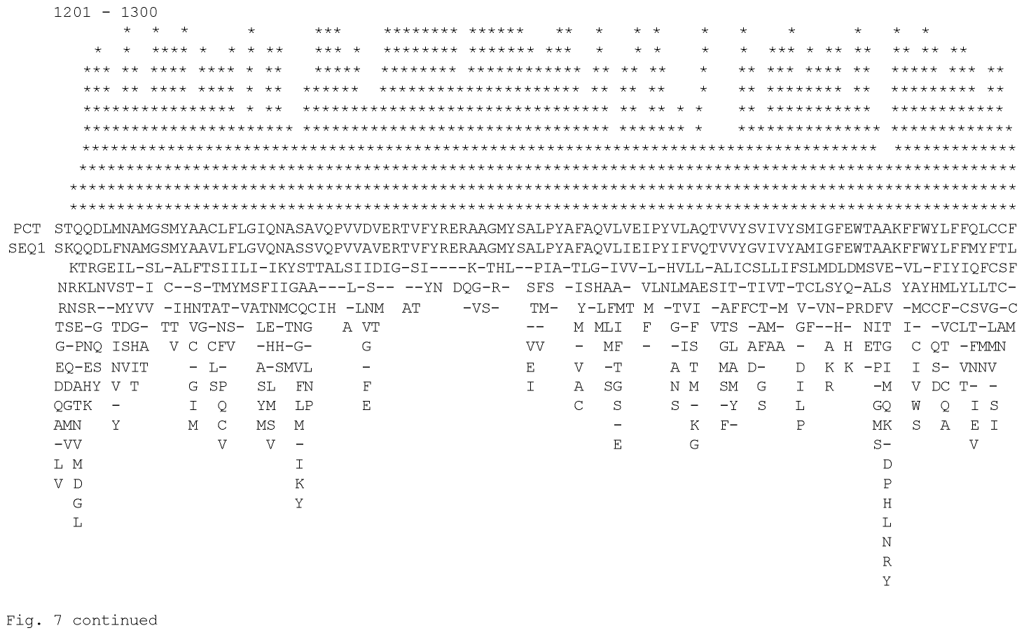

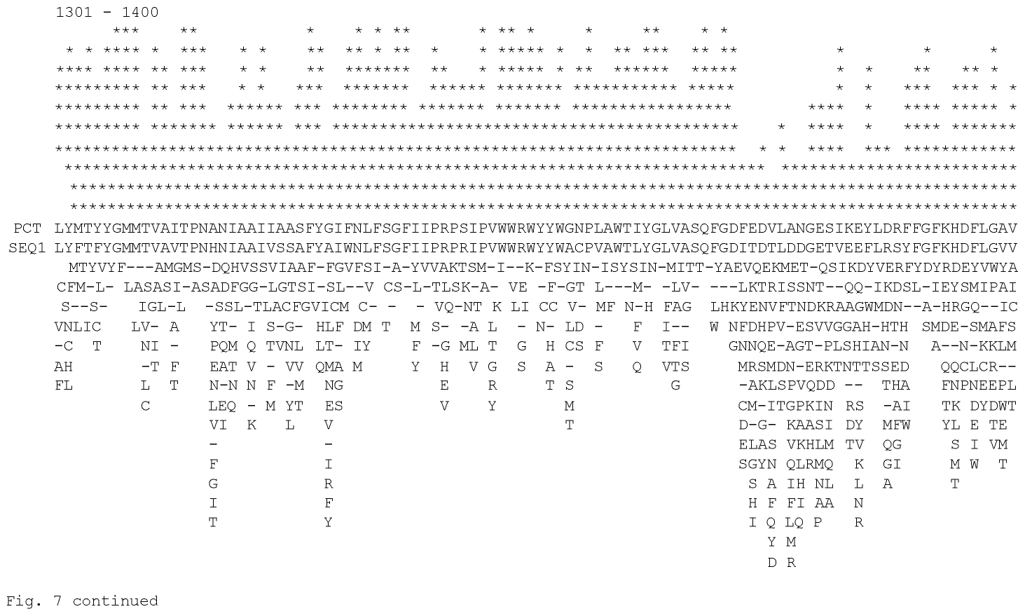

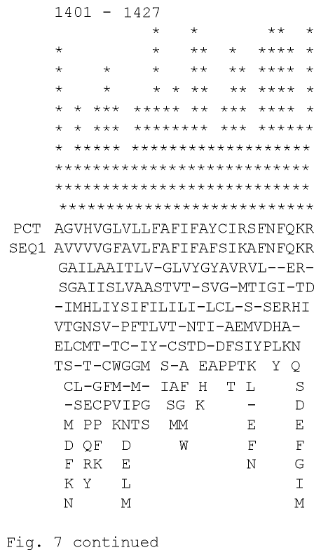

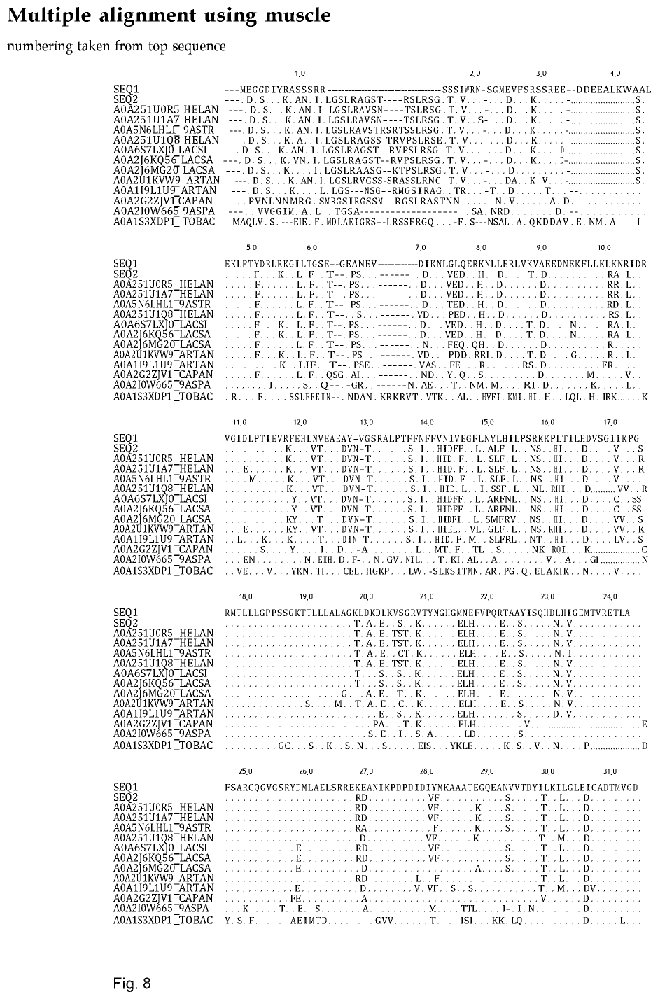

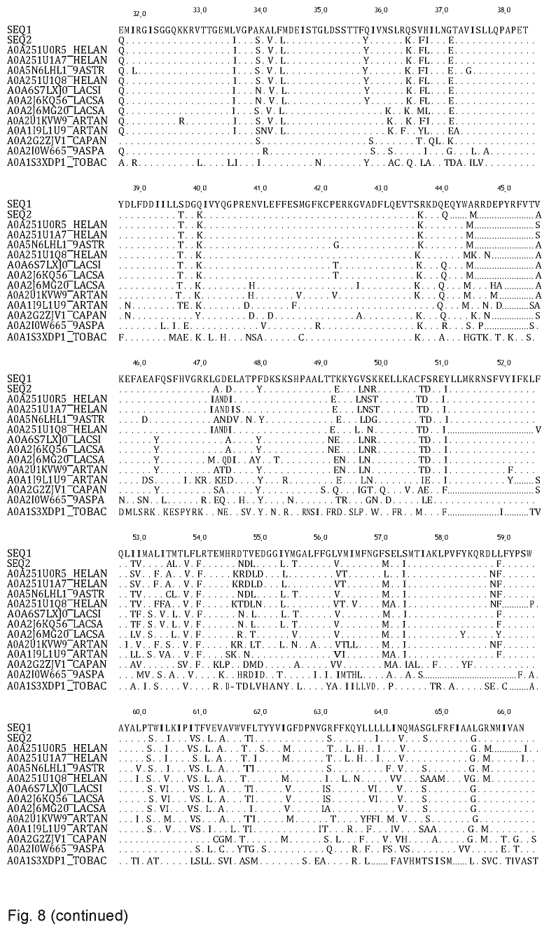

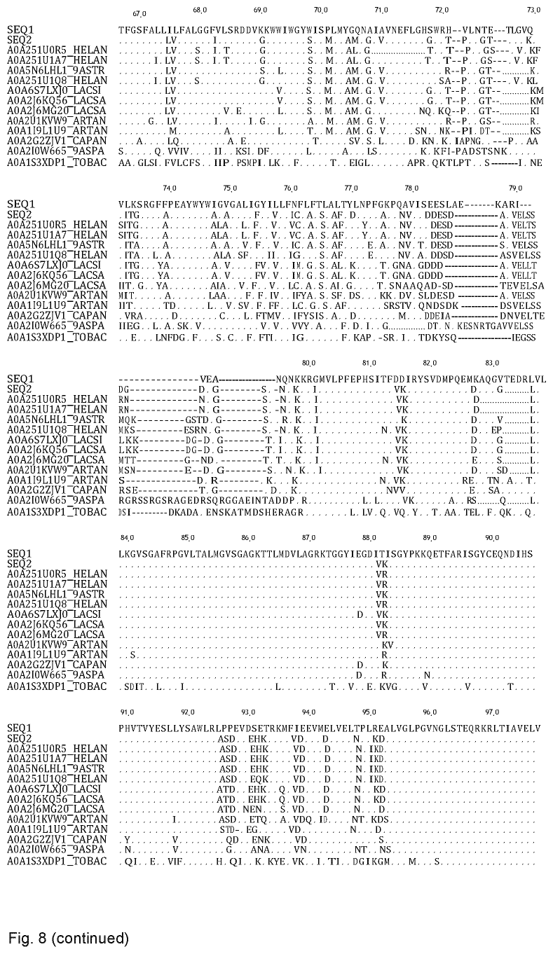

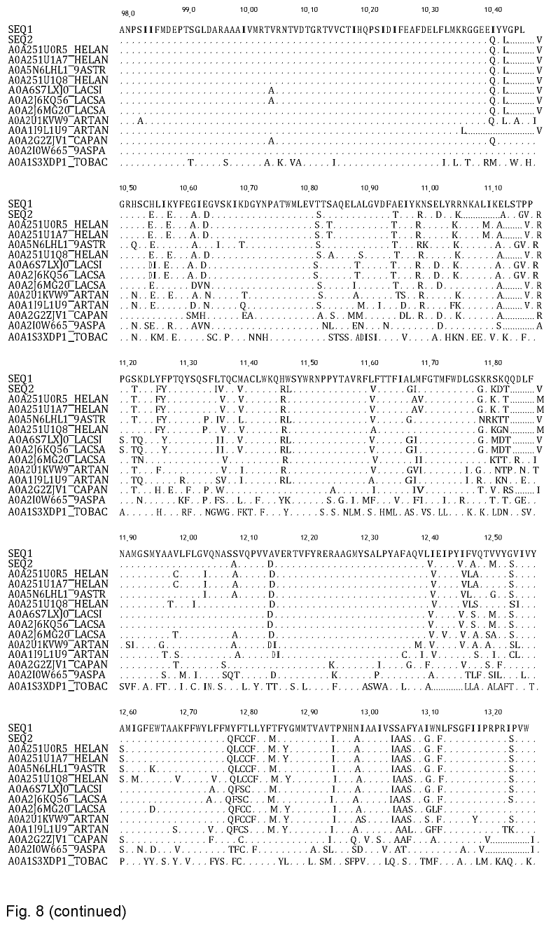

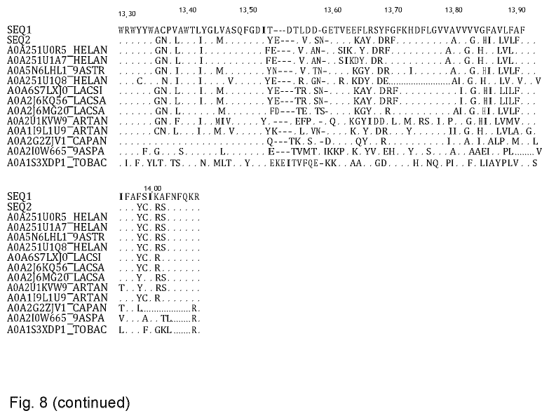

The sunflower PCT gene coding for A0A251U0R5_HELAN (legend in FIG. 5: “HaABC”) (control: AtPDR9) was coexpressed with different coumarin biosynthetic genes in leaves of N. benthamiana plants according to Example 7. Basal scopoletin and scoparone secretion was observed (cf. FIG. 5) upon expression of only F6H1 or F6H1+OMT3, respectively. Upon addition of Agrobacteria carrying the A0A251U0R5_HELAN construct, increased content of both coumarins could be detected in the leaf wash water (LWW), indicating transport of both coumarins onto the leaf by A0A251U0R5_HELAN

Note that upon coexpression of coumarin biosynthesis genes with the already published AtPDR9 transporter, no increased coumarin secretion was documented, indicating that AtPDR9 is not able to transport the antifungal coumarins scoploletin and scoparone.

The sunflower PCT gene coding for A0A251U0R5_HELAN (legend in FIG. 5: “HaABC”) was expressed in leaves of scopoletin-hyperaccumulating N. benthamiana plants (AtF6′H1 background) according to Example 7. We found that export of coumarins to the leaf surface is detectable by blue fluoresce on the leaf surface (not shown). Overexpression of the PCT gene (operably linked to a 35S promoter) resulted in five-fold increase of extracellular scopoletin compared to the p19 Agroinfiltration control (see FIG. 6A). Conditional expression of the PCT gene (A0A251U0R5_HELAN under control of a dexamethasone-inducible promoter) boosted the overall leaf content of coumarins (cf. FIG. 6B), presumably by accumulation of scopoletin on the leaf surface and in the apoplast. Germination of 1 mg/ml P. pachyrhizi spores in leaf washes (recollected droplets after 24 h, cf. Example 4) of N. benthamiana expressing A0A251U0R5_HELAN was reduced to less than 40% in comparison with the p19 Agroinfiltration control 24 h after DEX induction, see FIG. 6C.

### Example 11: Cloning of Overexpression Vector Constructs for Stable Soybean Transformation

The DNA sequence of the A0A251U0R5_HELAN CDS (c-HaABC), the corresponding wild type promoter (p-HaABC) and t-OCS (octopin.synthase terminator from Agrobacterium tumefaciens) mentioned in this application were generated by DNA synthesis (Geneart, Regensburg, Germany). The HaABC promoter was synthesized in a way that a Pacl re-striction site is located upstream of the promoter and an Ascl restriction site downstream of the promoter. The HaABC CDS was synthesized in a way that a Ascl restriction site is located in front of the start-ATG and an Sbfl restriction site downstream of the stop-codon and the t-OCS terminator was synthesized as a Sbfl/Fsel fragment. The p-HaABC::c-HaABC::t-OCS expression cassette was cloned traditionally (ligase) in a Pacl/Sbfl digested Gateway pENTRY-att1:3 vector (Invitrogen, Life Technologies, Carlsbad, California, USA) in a way that the full-length CDS fragment is located in sense direction between the HaABC promoter and the t-OCS terminator.

The F6H1 DNA was synthesized in a way that a Ascl re-striction site is located in front of the start-ATG and an Sbfl restriction site downstream of the stop-codon. The synthesized DNA was digested using the restriction enzymes Sbfl and Ascl (NEB Biolabs) and ligated in a Sbfl/Ascl digested Gateway pENTRY-att4:1 vector (Invitrogen, Life Technologies, Carlsbad, California, USA) in a way that the full-length fragment is located in sense direction between the parsley ubiquitin promoter and the Agrobacterium tumefaciens derived octopine synthase terminator (t-OCS). The PcUbi promoter regulates constitutive expression of the ubi4-2 gene (accession number X64345) of Petroselinum crispum (Kawalleck et al. 1993 Plant Molecular Biology 21(4):673-684).

To generate the binary plant transformation vector containing the above described PcUbi::F6H1::OCS and p-HaABC::c-HaABC::t-OCS expression cassettes, a LR reaction (Gateway system, Invitrogen, Life Technologies, Carlsbad, California, USA) was performed according to manufacturer's protocol. As target a binary pDEST vector was used which is composed of: (1) a spectinomycin/streptomycin resistance cassette for bacterial selection (2) a pVS1 origin for replication in Agrobacteria (3) a ColE1 origin of replication for stable maintenance in E. coli and (4) between the right and left border an AHAS selection under control of an AtAHASL-promoter. The recombination reaction was trans-formed into E. coli (DH5alpha), miniprepped and screened by specific restriction digestions. A positive clone from each vector construct was sequenced and submitted to soybean transformation.

### Example 12: Soybean Transformation

The expression vector constructs (see example 11) is transformed into soybean.

### 12.1 Sterilization and Germination of Soybean Seeds

Virtually any seed of any soybean variety can be employed in the method of the invention. A variety of soybean cultivar (including Jack, Williams 82, Jake, Stoddard, CD215 and Resnik) is appropriate for soybean transformation. Soybean seeds are sterilized in a chamber with a chlorine gas produced by adding 3.5 ml 12N HCl drop wise into 100 ml bleach (5.25% sodium hypochlorite) in a desiccator with a tightly fitting lid. After 24 to 48 hours in the chamber, seeds are removed and approximately 18 to 20 seeds are plated on solid GM medium with or without 5 μM 6-benzyl-aminopurine (BAP) in 100 mm Petri dishes. Seedlings without BAP are more elongated and roots develop especially secondary and lateral root formation. BAP strengthens the seedling by forming a shorter and stockier seedling.

Seven-day-old seedlings grown in the light (>100 μEinstein/m²s) at 25 degree C. are used for explant material for the three-explant types. At this time, the seed coat was split, and the epicotyl with the unifoliate leaves are grown to, at minimum, the length of the cotyledons. The epicotyl should be at least 0.5 cm to avoid the cotyledonary-node tissue (since soybean cultivars and seed lots may vary in the developmental time a description of the germination stage is more accurate than a specific germination time).

For inoculation of entire seedlings, see Method A (example 12.3. and 12.3.2) or leaf explants see Method B (example 12.3.3).

For method C (see example 12.3.4), the hypocotyl and one and a half or part of both cotyledons are removed from each seedling. The seedlings are then placed on propagation media for 2 to 4 weeks. The seedlings produce several branched shoots to obtain explants from. The majority of the explants originated from the plantlet growing from the apical bud. These explants are preferably used as target tissue.

### 12.2—Growth and Preparation of Agrobacterium Culture

Agrobacterium cultures are prepared by streaking Agrobacterium (e.g., A. tumefaciens or A. rhizogenes) carrying the desired binary vector (e.g. H. Klee. R. Horsch and S. Rogers 1987 Agrobacterium-Mediated Plant Transformation and its further Applications to Plant Biology; Annual Review of Plant Physiology Vol. 38:467-486) onto solid YEP growth medium YEP media: 10 g yeast extract. 10 g Bacto Peptone. 5 g NaCl. Adjust pH to 7.0, and bring final volume to 1 liter with H2O, for YEP agar plates add 20 g Agar, autoclave) and incubating at 25.degree C. until colonies appeared (about 2 days). Depending on the selectable marker genes present on the Ti or Ri plasmid, the binary vector, and the bacterial chromosomes, different selection compounds are to be used for A. tumefaciens and A. rhizogenes selection in the YEP solid and liquid media. Various Agrobacterium strains can be used for the transformation method.

After approximately two days, a single colony (with a sterile toothpick) is picked and 50 ml of liquid YEP is inoculated with antibiotics and shaken at 175 rpm (25° C.) until an OD₆₀₀ between 0.8-1.0 is reached (approximately 2 d). Working glycerol stocks (15%) for transformation are prepared and one-ml of Agrobacterium stock aliquoted into 1.5 ml Eppendorf tubes then stored at −80° C.

The day before explant inoculation, 200 ml of YEP are inoculated with 5 μl to 3 ml of working Agrobacterium stock in a 500 ml Erlenmeyer flask. The flask is shaken overnight at 25° C. until the OD₆₀₀ is between 0.8 and 1.0. Before preparing the soybean explants, the Agrobacteria ARE pelleted by centrifugation for 10 min at 5,500×g at 2020 C. The pellet is suspended in liquid CCM to the desired density (OD₆₀₀ 0.5-0.8) and placed at room temperature at least 30 min before use.

### 12.3—Explant Preparation and Co-Cultivation (Inoculation)

### 12.3.1 Method A:

Explant Preparation on the Day of Transformation. Seedlings at this time had elongated epicotyls from at least 0.5 cm but generally between 0.5 and 2 cm. Elongated epicotyls up to 4 cm in length are successfully employed. Explants are then prepared with: i) with or without some roots, ii) with a partial, one or both cotyledons, all preformed leaves are removed including apical meristem, and the node located at the first set of leaves is injured with several cuts using a sharp scalpel.

This cutting at the node not only induces Agrobacterium infection but also distributes the axillary meristem cells and damaged pre-formed shoots. After wounding and preparation, the explants are set aside in a Petri dish and subsequently co-cultivated with the liquid CCM/Agrobacterium mixture for 30 minutes. The explants are then removed from the liquid medium and plated on top of a sterile filter paper on 15×100 mm Petri plates with solid co-cultivation medium. The wounded target tissues are placed such that they are in direct contact with the medium.

### 12.3.2 Modified Method A: Epicotyl Explant Preparation

Soybean epicotyl segments prepared from 4 to 8 d old seedlings are used as explants for regeneration and transformation. Seeds of soybean are germinated in 1/10 MS salts or a similar composition medium with or without cytokinins for 4 to 8 d. Epicotyl explants are prepared by removing the cotyledonary node and stem node from the stem section. The epicotyl is cut into 2 to 5 segments. Especially preferred are segments attached to the primary or higher node comprising axillary meristematic tissue.

The explants are used for Agrobacterium infection. Agrobacterium AGL1 harboring a plasmid with the gene of interest (GOI) and the AHAS, bar or dsdA selectable marker gene is cultured in LB medium with appropriate antibiotics overnight, harvested and suspended in a inoculation medium with acetosyringone. Freshly prepared epicotyl segments are soaked in the Agrobacterium suspension for 30 to 60 min and then the explants were blotted dry on sterile filter papers. The inoculated explants are then cultured on a co-culture medium with L-cysteine and TTD and other chemicals such as acetosyringone for increasing T-DNA delivery for 2 to 4 d. The infected epicotyl explants are then placed on a shoot induction medium with selection agents such as imazapyr (for AHAS gene), glufosinate (for bar gene), or D-serine (for dsdA gene). The regenerated shoots are subcultured on elongation medium with the selective agent. For regeneration of transgenic plants, the segments are then cultured on a medium with cytokinins such as BAP, TDZ and/or Kinetin for shoot induction. After 4 to 8 weeks, the cultured tissues are transferred to a medium with lower concentration of cytokinin for shoot elongation. Elongated shoots are transferred to a medium with auxin for rooting and plant development. Multiple shoots are regenerated. Many stable transformed sectors showing strong cDNA expression are recovered. Soybean plants are regenerated from epicotyl explants. Efficient TDNA delivery and stable transformed sectors are demonstrated.

### 12.3.3 Method B: Leaf Explants

For the preparation of the leaf explant the cotyledon is removed from the hypocotyl. The cotyledons are separated from one another and the epicotyl is removed. The primary leaves, which consist of the lamina, the petiole, and the stipules, are removed from the epicotyl by carefully cutting at the base of the stipules such that the axillary meristems are included on the explant. To wound the explant as well as to stimulate de novo shoot formation, any pre-formed shoots are removed and the area between the stipules was cut with a sharp scalpel 3 to 5 times. The explants are either completely immersed or the wounded petiole end dipped into the Agrobacterium suspension immediately after explant preparation. After inoculation, the explants are blotted onto sterile filter paper to remove excess Agrobacterium culture and place explants with the wounded side in contact with a round 7 cm Whatman paper overlaying the solid CCM medium (see above). This filter paper prevents A. tumefaciens overgrowth on the soybean-explants. Wrap five plates with Parafilm.™. “M” (American National Can, Chicago, Ill., USA) and incubate for three to five days in the dark or light at 25° C.

### 12.3.4 Method C: Propagated Axillary Meristem

For the preparation of the propagated axillary meristem explant propagated 3-4 week-old plantlets are used. Axillary meristem explants can be pre-pared from the first to the fourth node. An average of three to four explants could be obtained from each seedling. The explants are prepared from plantlets by cutting 0.5 to 1.0 cm below the axillary node on the internode and removing the petiole and leaf from the explant. The tip where the axillary meristems lie is cut with a scalpel to induce de novo shoot growth and allow access of target cells to the Agrobacterium. Therefore, a 0.5 cm explant included the stem and a bud. Once cut, the explants are immediately placed in the Agrobacterium suspension for 20 to 30 minutes. After inoculation, the explants are blotted onto sterile filter paper to remove excess Agrobacterium culture then placed almost completely immersed in solid CCM or on top of a round 7 cm filter paper overlaying the solid CCM, depending on the Agrobacterium strain. This filter paper prevents Agrobacterium overgrowth on the soybean explants. Plates are wrapped with Parafilm.™. “M” (American National Can, Chicago, Ill., USA) and incubated for two to three days in the dark at 25° C.

### 12.4—Shoot Induction

After 3 to 5 days co-cultivation in the dark at 25° C., the explants are rinsed in liquid SIM medium (to remove excess Agrobacterium) (SIM, see Olhoft et al 2007 A novel Agrobacterium rhizogenes-mediated transformation method of soybean using primary-node explants from seedlings In Vitro Cell. Dev. Biol.—Plant (2007) 43:536-549; to remove excess Agrobacterium) or Modwash medium (1×B5 major salts, 1×B5 minor salts, 1×MSIII iron, 3% Sucrose, 1×B5 vitamins, 30 mM MES, 350 mg/L Timentin pH 5.6, WO 2005/121345) and blotted dry on sterile filter paper (to prevent damage especially on the lamina) before placing on the solid SIM medium. The approximately 5 explants (Method A) or 10 to 20 (Methods B and C) explants are placed such that the target tissue was in direct contact with the medium. During the first 2 weeks, the explants could be cultured with or without selective medium. Preferably, explants are transferred onto SIM without selection for one week.

For leaf explants (Method B), the explant should be placed into the medium such that it is perpendicular to the surface of the medium with the petiole imbedded into the medium and the lamina out of the medium.

For propagated axillary meristem (Method C), the explant is placed into the medium such that it is parallel to the surface of the medium (basipetal) with the explant partially embedded into the medium.

Wrap plates with Scotch 394 venting tape (3M, St. Paul, Minn., USA) are placed in a growth chamber for two weeks with a temperature averaging 25.degree. C. under 18 h light/6 h dark cycle at 70-100 μE/m²s. The explants remain on the SIM medium with or without selection until de novo shoot growth occurred at the target area (e.g., axillary meristems at the first node above the epicotyl). Transfers to fresh medium can occur during this time. Explants are transferred from the SIM with or without selection to SIM with selection after about one week. At this time, there is considerable de novo shoot development at the base of the petiole of the leaf explants in a variety of SIM (Method B), at the primary node for seedling explants (Method A), and at the axillary nodes of propagated explants (Method C).

Preferably, all shoots formed before transformation are removed up to 2 weeks after cocultivation to stimulate new growth from the meristems. This helped to reduce chimerism in the primary transformant and increase amplification of transgenic meristematic cells. During this time the explant may or may not be cut into smaller pieces (i.e. detaching the node from the explant by cutting the epicotyl).

### 12.5—Shoot Elongation

After 2 to 4 weeks (or until a mass of shoots is formed) on SIM medium (preferably with selection), the explants are transferred to SEM medium (shoot elongation medium, see Olhoft et al 2007 A novel Agrobacterium rhizogenes-mediated transformation method of soybean using primary-node explants from seedlings. In Vitro Cell. Dev. Biol.—Plant (2007) 43:536-549) that stimulates shoot elongation of the shoot primordia. This medium may or may not contain a selection compound.

After every 2 to 3 weeks, the explants are transferred to fresh SEM medium (preferably containing selection) after carefully removing dead tissue. The explants should hold together and not fragment into pieces and retain somewhat healthy. The explants are continued to be transferred until the explant dies or shoots elongate. Elongated shoots >3 cm are removed and placed into RM medium for about 1 week (Methods A and B), or about 2 to 4 weeks depending on the cultivar (Method C) at which time roots began to form. In the case of explants with roots, they are transferred directly into soil. Rooted shoots are transferred to soil and hardened in a growth chamber for 2 to 3 weeks before transferring to the greenhouse. Regenerated plants obtained using this method are fertile and produced on average 500 seeds per plant.

After 5 days of co-cultivation with Agrobacterium tumefaciens transient expression of the gene of interest (GOI) is widespread on the seedling axillary meristem explants especially in the regions wounding during explant preparation (Method A). Explants are placed into shoot induction medium without selection to see how the primary-node responds to shoot induction and regeneration. Thus far, greater than 70% of the explants were formed new shoots at this region. Expression of the GOI is stable after 14 days on SIM, implying integration of the T-DNA into the soybean genome. In addition, preliminary experiments results in the formation of cDNA expressing shoots forming after 3 weeks on SIM.

For Method C, the average regeneration time of a soybean plantlet using the propagated axillary meristem protocol is 14 weeks from explant inoculation. Therefore, this method has a quick regeneration time that leads to fertile, healthy soybean plants.

### Example 13: Pathogen Assay for Soybean

### 13.1. Growth of Plants

10 T1 soybean plants per event are potted and grown for 3-4 weeks in the phytochamber (16 h day-und 8 h-night-Rhythm at a temperature of 16° and 22° C. und a humidity of 75%) till the first 2 trifoliate leaves were fully expanded.

### 13.2 Inoculation

The plants are inoculated with spores of P. pachyrhizi. In order to obtain appropriate spore material for the inoculation, soybean leaves, which are infected with rust 15-20 days ago, are taken 2-3 days before the inoculation and transferred to agar plates (1% agar in H2O). The leaves are placed with their upper side onto the agar, which allowed the fungus to grow through the tissue and to produce very young spores. For the inoculation solution, the spores are knocked off the leaves and are added to a Tween-H2O solution. The counting of spores is performed under a light microscope by means of a Thoma counting chamber. For the inoculation of the plants, the spore suspension is added into a compressed-air operated spray flask and applied uniformly onto the plants or the leaves until the leaf surface is well moisturized. For macroscopic assays a spore density of 1−5×10⁵ spores/ml is used. For the microscopy, a density of >5×10⁵ spores/ml is used. The inoculated plants are placed for 24 hours in a greenhouse chamber with an average of 22° C. and >90% of air humidity. The following cultivation is performed in a chamber with an average of 25° C. and 70% of air humidity.

### Example 14: Microscopical Screening

For the Evaluation of the Pathogen Development, the inoculated leaves of plants are stained with aniline blue 48 hours after infection.

The aniline blue staining serves for the detection of fluorescent substances. During the defense reactions in host interactions and non-host interactions, substances such as phenols, callose or lignin accumulate or are produced and are incorporated at the cell wall either locally in papillae or in the whole cell (hypersensitive reaction, HR). Complexes are formed in association with aniline blue, which lead e.g. in the case of callose to yellow fluorescence. The leaf material is transferred to falcon tubes or dishes containing destaining solution II (ethanol/acetic acid 6/1) and is incubated in a water bath at 90° C. for 10-15 minutes. The destaining solution II is removed immediately thereafter, and the leaves are washed 2× with water. For the staining, the leaves are incubated for 1.5-2 hours in staining solution II (0.05% aniline blue=methyl blue, 0.067 M di-potassium hydrogen phosphate) and analyzed by microscopy immediately thereafter.

The different interaction types are evaluated (counted) by microscopy. An Olympus UV microscope BX61 (incident light) and a UV Longpath filter (excitation: 375/15, Beam splitter: 405 LP) are used. After aniline blue staining, the spores appeared blue under UV light. The papillae can be recognized beneath the fungal appressorium by a green/yellow staining. The hypersensitive reaction (HR) is characterized by a whole cell fluorescence

### Example 15: Evaluating the Susceptibility to Soybean Rust

The progression of the soybean rust disease is scored in percent by the estimation of the diseased area (area which was covered by sporulating uredinia) on the backside (abaxial side) of the leaf. Additionally, the yellowing of the leaf is taken into account. A scheme illustrating the disease rating can be found in WO2016124515 and WO2020120753.

At all 10 T1 soybean plants per event and 5-10 independent events per construct are inoculated with spores of Phakopsora pachyrhizi. The macroscopic disease symptoms of soybean against P. pachyrhizi of the inoculated soybean plants are scored 14 days after inoculation.

The average of the percentage of the leaf area showing fungal colonies or strong yellowing/browning on all leaves is considered as diseased leaf area. At all 10 T1 soybean plants per event and 5-10 independent events per construct T1 plants per construct (expression checked by RT-PCR) are evaluated in parallel to non-transgenic control plants. Non-transgenic soybean plants grown in parallel to the transgenic plants are used as controls.

The expression of the HaABC gene leads to enhanced resistance of soybean against Phakopsora pachyrhizi by secretion of coumarins (e.g. Scopoletin).

Secretion of coumarins by expression of the PCT coumarin transporter gene A0A251U0R5_HELAN leads to strong pathogen resistance with limited impact on the health of the plant.

### Example 16

Execution of experiments described above (see examples 1-14) replacing A0A251U0R5_HELAN with any other proven coumarin transporter, such as A0A251U1Q8_HELAN, A0A5N6LHL1_9ASTR, A0A119L1U9_ARTAN, A0A2J6KQ56_LACSA and A0A2G2ZJV1_CAPAN yields comparable results as described above (data not shown). All improvements described in this application are linked to the function of a certain protein to increase the transport antifungal coumarins such as, but not limited to, scopoletin and scoparone to the leaf surface. Therefore, exploiting any coumarin transporter or protein with similar function, but unrelated protein sequence, will lead to the improvements in agronomic performance described in this application, as long as it leads to the accumulation of antifungal coumarins on the leaf surface.

1. Plant cell comprising a nucleic acid for expression of a heterologous coumarin transporter (PCT) gene.

2. Plant cell according to claim 1, wherein the PCT gene codes for a PCT protein, wherein the PCT protein comprises, in InterPro-nomenclature and in N- to C-terminal sequence:
i) an IPR029481 ABC-transporter, N-terminal domain,
ii) an IPR003439 ABC transporter-like, ATP-binding domain,
iii) an IPR013525 ABC-2 type transporter domain,
iv) an IPR013581 Plant PDR ABC transporter associated domain,
v) an IPR034003 ATP-binding cassette transporter, PDR-like subfamily G, domain 2 domain, vi) a further IPR013525 ABC-2 type transporter domain.

3. Plant cell according to claim 1 wherein the PCT gene
a) codes for a PCT protein which has 20-90% sequence identity to SEQ ID NO. 1, and/or
b) is obtainable or obtained by selecting, from the genome of a plant capable of secreting coumarins, a gene having 20-90% sequence identity to SEQ ID NO. 1.

4. Plant cell according to claim 1, wherein the plant cell is a transgenic plant cell, and/or wherein the PCT gene is operably linked to a heterologous promoter and/or terminator.

5. Plant cell according to claim 1, comprising a metabolic pathway for production of one or more coumarins.

6. Plant cell according to claim 1, wherein expression of the PCT gene and/or of one or more genes, if present, of the metabolic pathway for production of one or more coumarins is/are directed in a way that expression occurs in root, stem and/or leaf cells.

7. Plant or plant part comprising a plant cell according to claim 1.

8. Plant or plant part according to claim 7, wherein the plant exhibits
coumarin accumulation on a surface of the plant or plant part, and/or
reduced, delayed or inhibited germination or growth of a phytopathogenic microorganism of a surface of the plant or plant part, and/or
increased resistance against infection by a phytopathogenic microorganism and/or increased resistance against parasitic plants,

9. Plant or plant part according to claim 7, wherein the plant, plant part or pant cell is soybean, beans, pea, clover, kudzu, lucerne, lentils, lupins, vetches, groundnut, rice, wheat, barley, arabidopsis, lentil, banana, canola, cotton, potato, maize, sugar cane, alfalfa, sugar beet, sunflower, rapeseed, sorghum, rice, cabbage, tomato, peppers, sugar cane and tobacco.

10. Plant progeny obtained by breeding a plant according to claim 7, wherein the progeny comprises the heterologous PCT gene.

11. Non-propagative plant part or material of a plant or plant part according claim 7.

12. Product of a plant, plant part or plant cell according to claim 1, wherein the product is obtainable or obtained by
i) collecting a material of said plant, plant part or plant cell, and
ii) disrupting the collected material.

13. Method for providing or increasing coumarin accumulation capability on a plant surface, comprising mutating a wild type gene such that in the correspondingly encoded PCT protein
the number of differences between the wild type gene sequence and the amino acid sequence SEQ ID NO. 2 is reduced, and/or
one or more or all of the following mutations, in the numbering according to SEQ ID NO. 2, are introduced into the wild type gene sequence: X8K, X10A, X31T, X32S, X39M, X40D, X67D, X75F, X76G, X78T, X80P, X81S, X84V, X87D, X88N, X91V, X92E, X95K, X96H, X106D, X111K, X118R, X120L, X125I, X127L, X138T, X142D, X156F, X157H, X1601, X164L, X165L, X170L, X171L, X172P, X174S, X178I, X182D, X212A, X218E, X221S, X224K, X233H, X241S, X308A, X310V, X332Q, X356K, X381Q, X382F, X383L, X387E, X416I, X419Q, X424N, X427E, X456E, X460M, X464E, X472K, X473E, X483V, X485R, X489N, X492A, X493T, X497K, X498S, X508E, X512L, X521C, X522T, X523D, X537I, X541F, X544S, X545V, X553V, X562R, X563D, X564L, X567G, X569L, X571M, X574L, X577G, X578V, X579T, X581I, X625S, X626F, X629A, X633T, X647T, X655I, X658L, X669F, X685S, X688L, X689L, X690L, X692F, X701R, X702D, X714S, X720A, X725A, X731G, X732H, X735R, X736T, X737P, X738L, X739N, X740G, X741S, X742T, X746F, X747S, X748I, X752G, X755A, X756E, X772L, X773V, X783A, X786N, X789G, X791A, X794N, X796S, X799D, X800E, X802D, X803A, X804A, X805V, X808T, X809S, X810R, X811N, X812N, X814G, X816Q, X834D, X836K, X847D, X856L, X900D, X947D, X950E, X951H, X952K, X957V, X958D, X9691, X980N, X1073E, X1080A, X1102T, X1108A, X1115E, X1116I, X1128A, X1131A, X1135V, X1144F, X1158V, X1200K, X1203Q, X1207M, X1215A, X1233D, X1258V, X1262I, X1271V, X1287A, X1296Q, X1297L, X1298C, X1303M, X1312A, X1317A, X1318N, X1321A, X13301, X1339I, X1340P, X1354G, X1372F, X1374D, X1375V, X1378N, X1384E, X1385Y, X1387D, X1390F, X1400V, X1402G, X1404H, X1405V, X1407L, X1408V, X1409L, X1413F, X1417Y.

14. Automated plant selection method, comprising the steps of
i) obtaining, for each seed of a plurality of seeds, a sample comprising genetic material of a tissue body representative for said seed,
ii) determining the presence of a PCT gene according to claim 1, in the genetic material, and optionally the presence of one or more genes of a metabolic pathway for production of one or more coumarins,
iii) selecting those seed where the determination in step ii) gave a positive result.

15. (canceled)

16. The plant cell according to claim 3, wherein the PCT gene codes for a PCT protein which comprises one or more of the following mutations in the numbering according to SEQ ID NO. 2: X8K, X10A, X31T, X32S, X39M, X40D, X67D, X75F, X76G, X78T, X80P, X81S, X84V, X87D, X88N, X91V, X92E, X95K, X96H, X106D, X111K, X118R, X120L, X125I, X127L, X138T, X142D, X156F, X157H, X160I, X164L, X165L, X170L, X171L, X172P, X174S, X178I, X182D, X212A, X218E, X221S, X224K, X233H, X241S, X308A, X310V, X332Q, X356K, X381Q, X382F, X383L, X387E, X416I, X419Q, X424N, X427E, X456E, X460M, X464E, X472K, X473E, X483V, X485R, X489N, X492A, X493T, X497K, X498S, X508E, X512L, X521C, X522T, X523D, X537I, X541F, X544S, X545V, X553V, X562R, X563D, X564L, X567G, X569L, X571M, X574L, X577G, X578V, X579T, X581I, X625S, X626F, X629A, X633T, X647T, X655I, X658L, X669F, X685S, X688L, X689L, X690L, X692F, X701R, X702D, X714S, X720A, X725A, X731G, X732H, X735R, X736T, X737P, X738L, X739N, X740G, X741S, X742T, X746F, X747S, X748I, X752G, X755A, X756E, X772L, X773V, X783A, X786N, X789G, X791A, X794N, X796S, X799D, X800E, X802D, X803A, X804A, X805V, X808T, X809S, X810R, X811N, X812N, X814G, X816Q, X834D, X836K, X847D, X856L, X900D, X947D, X950E, X951H, X952K, X957V, X958D, X969I, X980N, X1073E, X1080A, X1102T, X1108A, X1115E, X1116I, X1128A, X1131A, X1135V, X1144F, X1158V, X1200K, X1203Q, X1207M, X1215A, X1233D, X1258V, X1262I, X1271V, X1287A, X1296Q, X1297L, X1298C, X1303M, X1312A, X1317A, X1318N, X1321A, X1330I, X1339I, X1340P, X1354G, X1372F, X1374D, X1375V, X1378N, X1384E, X1385Y, X1387D, X1390F, X1400V, X1402G, X1404H, X1405V, X1407L, X1408V, X1409L, X1413F, X1417Y.
